SEQUENCE LISTING (32 sequences)

SEQ ID NO: 1 - DNA, 1479 bp, Human immunodeficiency virus type 1
atgggtgcgagagcgtcaatattaagaggggaaaaattagataaatgggaaaaaattaggttaaggccagggggaaagaaacattatatgttaaaacacatagtatgggcaagcagggagctggaaagatttgcacttaaccctggccttttagaaacatcagaaggatgtaaacaaataatgaaacagctacaaccagctctccagacaggaacagaggaacttaaatcattatacaacacagtagcaactctctattgtgtacatgaaaagatagaagtacgagacaccaaggaagccttagataagatagaggaagaacaaaacaaatgtcagcaaaaaacgcagcaggcaaaagcggctgacgggaaagtcagtcaaaattatcctatagtgcagaatctccaagggcaaatggtacatcaagccatatcacctagaaccttgaatgcatgggtaaaagtaatagaagaaaaggcttttagcccagaggtaatacccatgtttacagcattatcagaaggagccaccccacaagatttaaacaccatgttaaatacagtggggggacatcaagcagccatgcaaatgttaaaagatactattaatgaagaggctgcagaatgggatagagtacatccagtccatgcggggcctattgcaccaggccagatgagagaaccaaggggaagtgacatagcaggaactactagtacccttcaggaacaaatagcatggatgacaagtaacccacctattccagtgggagacatctataaaagatggataattctggggttaaataaaatagtgagaatgtatagcccggtcagcattttggacataagacaagggccaaaggaaccctttcgagactatgtagatcggttctttaaaactttaagagctgaacaagctacacaagaagtaaaaaattggatgacagacaccttgttagtccaaaatgcgaacccagattgtaagaccattttgagagcattaggaccaggggctacattagaagaaatgatgacagcatgtcaaggggtgggaggacctggtcacaaagcaagagtattggctgaggcaatgagtcaagcaaacagtggaaacataatgatgcagagaagcaattttaaaggccctagaagaattgttaaatgttttaactgtggcaaggaagggcacatagccagaaattgcagagcccctaggaaaaaaggctgttggaaatgtggaaaggaaggacaccaaatgaaagactgtactgaaaggcaggctaattttttagggaaaatttggccttcccacaaggggaggccagggaatttccttcagaacagaccagagccaacagccccaccagcagagagcttcaggttcgaagagacaacccccgctccgaaacaggagccgatagaaagggaacccttaacttccctcaaatcactctttggcagcgaccccttgtctcaataa

SEQ ID NO: 2 - protein, 492 aa, Human immunodeficiency virus type 1
MGARASILRGEKLDKWEKIRLRPGGKKHYMLKHIVWASRELERFALNPGLLETSEGCKQIMKQLQPALQTGTEELKSLYNTVATLYCVHEKIEVRDTKEALDKIEEEQNKCQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRVHPVHAGPIAPGQMREPRGSDIAGTTSTLQEQIAWMTSNPPIPVGDIYKRWIILGLNKIVRMYSPVSILDIRQGPKEPFRDYVDRFFKTLRAEQATQEVKNWMTDTLLVQNANPDCKTILRALGPGATLEEMMTACQGVGGPGHKARVLAEAMSQANSGNIMMQRSNFKGPRRIVKCFNCGKEGHIARNCRAPRKKGCWKCGKEGHQMKDCTERQANFLGKIWPSHKGRPGNFLQNRPEPTAPPAESFRFEETTPAPKQEPIEREPLTSLKSLFGSDPLSQ

SEQ ID NO: 3 - DNA, 2997 bp, Human immunodeficiency virus type 1
tttagggaaaatttggccttcccacaaggggaggccagggaatttccttcagaacagaccagagccaacagccccaccagcagagagcttcaggttcgaagaaacaacccccgctccgaaacaggagccgagagaaagggaacccttaacttccctcaaatcactctttggcagcgaccccttgtctcaataaaaatagggggccagacaagggaggctctcttagacacaggagcagatgatacagtattagaagacataaatttgccaggaaaatggaaaccaaaaatgataggaggaattggaggttttatcaaagtaagacagtatgatcaaatacttatagaaatttgtggaaaaaaggctataggtacagtattagtagggcctacacctgtcaacataattggcagaaacatgttgactcagcttggatgcacactaaactttccaatcagtcccattgaaactgtaccagtaaaactgaagccaggaatggatggcccaaaggttaaacaatggccgttaacagaagagaaaataaaagcattaacagcaatttgtgaagaaatggaaaaggaaggaaaaattacaaaaattgggcctgaaaatccatataacactccaatatttgccataaaaaagaaagacagcactaagtggagaaaattagtagatttcagggaactcaataaaagaactcaagacttttgggaggttcaattaggaataccacacccagcagggttaaaaaagaaaaaatcagtgacagtactggatgtgggagatgcatatttttcagttcctttagatgaaggcttcaggaaatatactgcattcaccatacctagtataaacaatgaaacaccagggattagatatcaatataatgtgcttccacaaggatggaaagggtcaccagcaatattccagggtagcatgacaaaaatcttagagccctttagagctcaaaatccagaaatagtcatctatcaatatatggatgacttgtatgtaggatctgacttagaaatagggcaacatagagcaaaaatagaagagttaagagaacatctattaaagtggggatttaccacaccagacaaaaaacatcagaaagaacccccatttctttggatggggtatgaactccatcctgacaaatggacagtacagcctatacagctgccagaaaaggatagctggactgtcaatgatatacagaagttagtgggaaaattaaactgggcaagtcagatttacccagggattaaagtaaggcaactttgtaagctccttagggggaccaaagcactaacagacatagtaccactaactgaagaagcagaattagaattggcagagaacagggaaattctaaaagaaccagtgcatggagtatattatgacccatcaaaagacttgatagctgaaatacagaaacagggggatgaccaatggacatatcaaatttaccaagaaccattcaaaaacctgaagacaggaaagtatgcaaaaaggaggactacccacactaatgatgtaaaacagttaacagaggcagtgcaaaaaatatccttggaaagcatagtaatatggggaaagactcctaaatttagactacccatccaaaaagaaacatgggaaatatggtggacagactattggcaagccacatggattcctgagtgggagtttgttaatacccctcccctagtaaaactatggtaccagctagaaaaagaacccatagcaggagcagaaactttctatgtagatggagcagctaatagggaaactaaaataggaaaagcggggtatgttactgacagaggaaggcagaaaattgtaactctaagtgaaacaacaaatcagaagactgaattacaagcaattcagctagctttgcaagattcagaatcagaagtaaacataataacagactcacagtacgcattaggaatcattcaagcacaaccagataggagtgaatcagagttggtcaatcaaataatagaacaattaataaaaaaggaaagggtctatctgtcatgggtaccagcacacaacggacttgcaggaaatgaacatgtagataaattagtaagtaggggaatcaggaaagtgctggttctagatggaatagataaggctcatgaagagcatgaaaagtatcacagcaattggagagcaatggctagtgagtttaatctgccacccgtagtagcaagagaaatagtagccagctgtgataaatgtcagctaaaaggggaagccatacatggacaagtagattgtagtccggggatatggcaattagattgtacacatttagaaggaaaaatcatcctggtagcagtccatgtagccagtggctacatagaagcagaggttatcccagcagaaacaggacaagaaacagcatactatatactaaaattagcaggaagatggccagtcaaagtaatacatacagacaatggcagtaatttcaccagtgctgcagttaaggcagcctgttggtgggcaggtatccaacaggaatttgggattccctacaatccccaaagtcagggagtagtagaatccatgaataaagaattaaagaaaatcatagggcaggtaagagatcaagctgagcaccttaagacagcagtacaaatggcagtattcattcacaattttaaaagaaaaggggggattggggggtacagtgcaggggaaagaataatagacataatagcaacagacatacaaactaaagaattacaaaaacaaattataaaaattcaaaattttcgggtttattacagagacagcagagatcctatttggaaaggaccagccaagctactctggaaaggtgaaggggcagtagtaatacaagacaacagtgacataaaggtagtaccaaggaggaaagtaaaaatcattagggactatggaaaacagatggcaggtgctgattgtgtggcaggtagacaggatgaagattag

SEQ ID NO: 4 - protein, 998 aa, Human immunodeficiency virus type 1
FRENLAFPQGEAREFPSEQTRANSPTSRELQVRRNNPRSETGAERKGTLNFPQITLWQRPLVSIKIGGQTREALLDTGADDTVLEDINLPGKWKPKMIGGIGGFIKVRQYDQILIEICGKKAIGTVLVGPTPVNIIGRNMLTQLGCTLNFPISPIETVPVKLKPGMDGPKVKQWPLTEEKIKALTAICEEMEKEGKITKIGPENPYNTPIFAIKKKDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQGSMTKILEPFRAQNPEIVIYQYMDDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGTKALTDIVPLTEEAELELAENREILKEPVHGVYYDPSKDLIAEIQKQGDDQWTYQIYQEPFKNLKTGKYAKRRTTHTNDVKQLTEAVQKISLESIVIWGKTPKFRLPIQKETWEIWWTDYWQATWIPEWEFVNTPPLVKLWYQLEKEPIAGAETFYVDGAANRETKIGKAGYVTDRGRQKIVTLSETTNQKTELQAIQLALQDSESEVNIITDSQYALGIIQAQPDRSESELVNQIIEQLIKKERVYLSWVPAHNGLAGNEHVDKLVSRGIRKVLVLDGIDKAHEEHEKYHSNWRAMASEFNLPPVVAREIVASCDKCQLKGEAIHGQVDCSPGIWQLDCTHLEGKIILVAVHVASGYIEAEVIPAETGQETAYYILKLAGRWPVKVIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIRDYGKQMAGADCVAGRQDED

SEQ ID NO: 5 - DNA, 2535 bp, Human immunodeficiency virus type 1
atgagagtgatggggatacagaggaattggccacaatggtggatatggggcaccttaggcttttggatgataataatttgtagggtggtggggaacttgaacttgtgggtcacagtctattatggggtacctgtgtggaaagaagcaaaaactactctattctgtgcatcagatgctaaagcatatgataaagaagtacataatgtctgggctacacatgcctgtgtacccacagaccccaacccacgagaaatagttttggaaaatgtaacagaaaattttaacatgtggaaaaatgacatggtggatcagatgcatgaggatataatcagtttatgggatcaaagcctaaaaccatgtgtaaagttgaccccactctgtgtcactttaaattgtacaaatgcacctgcctacaataatagcatgcatggagaaatgaaaaattgctctttcaatacaaccacagagataagagataggaaacagaaagcgtatgcacttttttataaacctgatgtagtgccacttaataggagagaagagaataatgggacaggagagtatatattaataaattgcaattcctcaaccataacacaagcctgtccaaaggtcacttttgacccaattcctatacattattgtgctccagctggttatgcgattctaaagtgtaataataagacattcaatgggacaggaccatgcaataatgtcagcacagtacaatgtacacatggaattatgccagtggtatcaactcaattactgttaaatggtagcctagcagaagaagagataataattagatctgaaaatctgacaaacaatatcaaaacaataatagtccaccttaataaatctgtagaaattgtgtgtacaagacccaacaataatacaagaaaaagtataaggataggaccaggacaaacattctatgcaacaggtgaaataataggaaacataagagaagcacattgtaacattagtaaaagtaactggaccagtactttagaacaggtaaagaaaaaattaaaagaacactacaataagacaatagaatttaacccaccctcaggaggggatctagaagttacaacacatagctttaattgtagaggagaatttttctattgcaatacaacaaaactgttttcaaacaacagtgattcaaacaacgaaaccatcacactcccatgcaagataaaacaaattataaacatgtggcagaaggtaggacgagcaatgtatgcccctcccattgaaggaaacataacatgtaaatcaaatatcacaggactactattgacacgtgatggaggaaagaatacaacaaatgagatattcagaccgggaggaggaaatatgaaggacaattggagaagtgaattatataaatataaagtggtagaaattgagccattgggagtagcacccactaaatcaaaaaggagagtggtggagagagaaaaaagagcagtgggactaggagctgtactccttgggttcttgggagcagcaggaagcactatgggcgcggcgtcaataacgctgacggtacaggccagacaactgttgtctggtatagtgcaacagcaaagcaatttgctgagagctatagaggcgcaacagcatatgttgcaactcacggtctggggcattaagcagctccagacaagagtcttggctatagagagatacctaaaggatcaacagctcctagggctttggggctgctctggaaaaatcatctgcaccactgctgtgccttggaactccagttggagtaataaatctcaagaagatatttgggataacatgacctggatgcagtgggatagagaaattagtaattacacaggcacaatatataggttacttgaagactcgcaaaaccagcaggagaaaaatgaaaaagatttattagcattggacagttggaaaaacttgtggaattggtttaacataacaaattggctgtggtatataaaaatattcatcatgatagtaggaggcttgataggtttgagaataatttttggtgtactcgctatagtgaaaagagttaggcagggatactcacctttgtcgtttcagacccttaccccaagcccgaggggtcccgacaggctcggaagaatcgaagaagaaggtggagagcaagacaaagacagatccattcgattagtgagcggattcttagcacttgcctgggacgatctgcggagcctgtgcctcttcagctaccaccacttgagagacttcatattgattgcagcgagagcagcggaacttctgggacgcagcagtctcaggggactgcagagagggtgggaagcccttaagtatctgggaaatcttgtgcagtatgggggtctggagctaaaaagaagtgctattaaactgtttgataccatagcaatagcagtagctgaaggaacagataggattcttgaagtaatacagagaatttgtagagctatccgccacatacctataagaataagacagggctttgaagcagctttgcaataa

SEQ ID NO: 6 - protein, 844 aa, Human immunodeficiency virus type 1
MRVMGIQRNWPQWWIWGTLGFWMIIICRVVGNLNLWVTVYYGVPVWKEAKTTLFCASDAKAYDKEVHNVWATHACVPTDPNPREIVLENVTENFNMWKNDMVDQMHEDIISLWDQSLKPCVKLTPLCVTLNCTNAPAYNNSMHGEMKNCSFNTTTEIRDRKQKAYALFYKPDVVPLNRREENNGTGEYILINCNSSTITQACPKVTFDPIPIHYCAPAGYAILKCNNKTFNGTGPCNNVSTVQCTHGIMPVVSTQLLLNGSLAEEEIIIRSENLTNNIKTIIVHLNKSVEIVCTRPNNNTRKSIRIGPGQTFYATGEIIGNIREAHCNISKSNWTSTLEQVKKKLKEHYNKTIEFNPPSGGDLEVTTHSFNCRGEFFYCNTTKLFSNNSDSNNETITLPCKIKQIINMWQKVGRAMYAPPIEGNITCKSNITGLLLTRDGGKNTTNEIFRPGGGNMKDNWRSELYKYKVVEIEPLGVAPTKSKRRVVEREKRAVGLGAVLLGFLGAAGSTMGAASITLTVQARQLLSGIVQQQSNLLRAIEAQQHMLQLTVWGIKQLQTRVLAIERYLKDQQLLGLWGCSGKIICTTAVPWNSSWSNKSQEDIWDNMTWMQWDREISNYTGTIYRLLEDSQNQQEKNEKDLLALDSWKNLWNWFNITNWLWYIKIFIMIVGGLIGLRIIFGVLAIVKRVRQGYSPLSFQTLTPSPRGPDRLGRIEEEGGEQDKDRSIRLVSGFLALAWDDLRSLCLFSYHHLRDFILIAARAAELLGRSSLRGLQRGWEALKYLGNLVQYGGLELKRSAIKLFDTIAIAVAEGTDRILEVIQRICRAIRHIPIRIRQGFEAALQ

SEQ ID NO: 7 - DNA, 1905 bp, Human immunodeficiency virus type 1
gggggatgtgctgcaaggcgattaagttgggtaacgccagggttttcccaatcacgacgttgtaaaacgacagccaatgaattgaagcttatggctgctcgcgcatctatcctcagaggcgaaaagttggataagtgggaaaaaatcagactcaggccaggaggtaaaaaacactacatgctgaagcatatcgtgtgggcatctagggagttggagagatttgcactgaaccccggactgctggaaacctcagagggctgtaagcaaatcatgaaacagctccaaccagccttgcagaccggaacagaagagctgaagtccctttacaataccgtggcaaccctctattgcgtccacgagaagatcgaggtgagagacacaaaggaggccctggacaaaatcgaggaggagcagaataagtgccagcagaagacccagcaggcaaaggctgctgacggaaaggtctctcagaactatcctatcgttcagaaccttcaggggcagatggtgcaccaagcaatcagccctagaaccctgaacgcatgggtgaaggtgatcgaggagaaagccttttctcccgaggttatccccatgtttaccgccctgagcgaaggcgccactcctcaagacctgaacactatgctgaacacagtgggaggacaccaggccgctatgcagatgttgaaggataccatcaacgaggaggcagccgaatgggaccgcctccaccccgtgcacgccggacctatcgcccccggacaaatgagagaacctcgcggaagtgatattgccggtactaccagcacccttcaagagcagattgcttggatgaccagcaacccacccatcccagtgggcgatatttacaaaaggtggattattctggggctgaacaaaattgtgagaatgtactcccccgtctccatcctcgacatccgccaaggacccaaggagccttttagggattacgtggacagattcttcaaaacccttagagctgagcaagccactcaggaggttaagaactggatgacagatactctgctcgtgcaaaacgctaaccccgattgcaaaaccatcttgagagctctcggtccaggtgccacccttgaggaaatgatgacagcatgtcaaggcgtgggaggacctgggcacaaggccagagttctcgctgaggccatgagccagacaaactcaggcaatatcatgatgcagaggagtaactttaagggtcccaggagaatcgtcaagtgcttcaattgtggcaaggagggtcacattgccaggaactgccgcgcccccaggaagaaaggctgctggaagtgtggcaaagagggccaccagatgaaggattgcaccgagcgccaagcaaacttcctgggaaagatttggcccagtcataagggccgccctggcaacttccttcaaaacagacccgagcctaccgccccccccgctgagtctttcagatttgaggagaccacccccgctccaaagcaggagccaattgagagagagcctctcaccagtctcaaaagcctctttggtagcgaccccctcagccaataagaattctagcttggcgtaatcatggtcatagctgtttcctgtgtgaaattgttatcagctcacaattccacacaacatacgagccggaagcataaagtgtaaagcctgggatgcctaatgagtgagctaactcacattagttgcgttgcgctcactgcccgctttccagtcgggaaacctgtcgtgccagctccattagtgaatcgtccaacgcacggggagaggcggtttgcgtattgggcgcacttccgcttcctcgctcactgactcgctgcgctcgttcgttcggctgcggcgagccgtatcagctcactcaaaggcggtaatacggttatc

SEQ ID NO: 8 - protein, 631 aa, Human immunodeficiency virus type 1
GGCAARRLSWVTPGFSQSRRCKTTANELKLMAARASILRGEKLDKWEKIRLRPGGKKHYMLKHIVWASRELERFALNPGLLETSEGCKQIMKQLQPALQTGTEELKSLYNTVATLYCVHEKIEVRDTKEALDKIEEEQNKCQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIRQGPKEPFRDYVDRFFKTLRAEQATQEVKNWMTDTLLVQNANPDCKTILRALGPGATLEEMMTACQGVGGPGHKARVLAEAMSQTNSGNIMMQRSNFKGPRRIVKCFNCGKEGHIARNCRAPRKKGCWKCGKEGHQMKDCTERQANFLGKIWPSHKGRPGNFLQNRPEPTAPPAESFRFEETTPAPKQEPIEREPLTSLKSLFGSDPLSQEFLGVIMVIAVSCVKLLSAHNSTQHTSRKHKVSLGCLMSELTHISCVALTARFPVGKPVVPAPLVNRPTHGERRFAYWAHFRFLAHLAALVRSAAASRISSLKGGNTVI

SEQ ID NO: 9 - DNA, 2577 bp, Human immunodeficiency virus type 1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctccttcccacaagggccggccaggcaatttccttcagaacagaccagagccaacagccccaccagcagagagcttcaggttcgaagagacaacccccgctccgaaacaggagccgagagaaagggaacccttaacttccctcaaatcactctttggcagcgaccccttgtctcaataaaaatcggcggccagacccgggaggccctgctggacaccggcgccgacgacaccgtgctggaggacatcaacctgcccggcaagtggaagcccaagatgatcggcggcatcggcggcttcatcaaggtgcggcagtacgaccagatcctgatcgagatctgcggcaagaaggccatcggcaccgtgctggtgggccccacccccgtgaacatcatcggccggaacatgctgacccagctgggctgcaccctgaacttccccatcagccccatcgagaccgtgcccgtgaagctgaagcccggcatggacggccccaaggtgaagcagtggcccctgaccgaggtgaagatcaaggccctgaccgccatctgcgaggagatggagaaggagggcaagatcaccaagatcggccccgagaacccctacaacacccccatcttcgccatcaagaaggaggacagcaccaagtggcggaagctggtggacttccgggagctgaacaagcggacccaggacttctgggaggtgcagctgggcatcccccaccccgccggcctgaagaagaagaagagcgtgaccgtgctggacgtgggcgacgcctacttcagcgtgcccctggacgagggcttccggaagtacaccgccttcaccatccccagcatcaacaacgagacccccggcatccggtaccagtacaacgtgctgccccagggctggaagggcagccccgccatcttccaggccagcatgaccaagatcctggagcccttccgggccaagaaccccgagatcgtgatctaccagtacatggccgccctgtacgtgggcagcgacctggagatcggccagcaccgggccaagatcgaggagctgcgggagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctggtgggcaagctgaactggaccagccagatctaccccggcatcaaggtgcggcagctgtgcaagctgctgcggggcaccaaggccctgaccgacatcgtgcccctgaccgaggaggccgagctggagctggccgagaaccgggagatcctgaaggagcccgtgcacggcgtgtactacgaccccagcaaggacctgatcgccgagatccagaagcagggcgacgaccagtggacctaccagatctaccaggagcccttcaagaacctgaaaaccggcaagtacgccaagcggcggaccacccacaccaacgacgtgaagcagctgaccgaggccgtgcagaagatcagcctggagagcatcgtgacctggggcaagacccccaagttccggctgcccatccagaaggagacctgggagatctggtggaccgactactggcaggccacctggatccccgagtgggagttcgtgaacaccccccccctggtgaagctgtggtaccagctggagaaggagcccatcgccggcgccgagaccttctacgtggacggcgccgccaaccgggagaccaagatcggcaaggccggctacgtgaccgaccggggccggcagaagatcgtgaccctgagcgagaccaccaaccagaaaaccgagctgcaggccatccagctggccctgcaggacagcgagagcgaggtgaacatcgtgaccgacagccagtacgccctgggcatcatccaggcccagcccgaccggagcgagagcgagctggtgaaccagatcatcgagcagctgatcaagaaggagcgggcctacctgagctgggtgcccgcccacaagggcatcggcggcgacgagcaggtggacaagctggtgagcagcggcatccggaaggtgctgtgatctagagaattc

SEQ ID NO: 10 - protein, 850 aa, Human immunodeficiency virus type 1
SRVSVMTVKTSDTCSSRRRSQLVCKRMPGADKPVRARQRVLAGVGAGLTMRHQSRLYECTICGVKYRTDAGENTASGAIRHSGCATVGKGDRCGPLRYYASWRKGDVLQGDVGRQGFPSHDVVKRRPVPSLHACRSTLEDPRVPSSFPQGPARQFPSEQTRANSPTSRELQVRRDNPRSETGAERKGTLNFPQITLWQRPLVSIKIGGQTREALLDTGADDTVLEDINLPGKWKPKMIGGIGGFIKVRQYDQILIEICGKKAIGTVLVGPTPVNIIGRNMLTQLGCTLNFPISPIETVPVKLKPGMDGPKVKQWPLTEVKIKALTAICEEMEKEGKITKIGPENPYNTPIFAIKKEDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQASMTKILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWTSQIYPGIKVRQLCKLLRGTKALTDIVPLTEEAELELAENREILKEPVHGVYYDPSKDLIAEIQKQGDDQWTYQIYQEPFKNLKTGKYAKRRTTHTNDVKQLTEAVQKISLESIVTWGKTPKFRLPIQKETWEIWWTDYWQATWIPEWEFVNTPPLVKLWYQLEKEPIAGAETFYVDGAANRETKIGKAGYVTDRGRQKIVTLSETTNQKTELQAIQLALQDSESEVNIVTDSQYALGIIQAQPDRSESELVNQIIEQLIKKERAYLSWVPAHKGIGGDEQVDKLVSSGIRKVL

SEQ ID NO: 11 - DNA, 2564 bp, Human immunodeficiency virus type 1
aagcttatgagggttatggggattcagagaaactggcctcagtggtggatttgggggacattgggattttggatgatcatcatctgtcgcgtcgtgggcaacctgaacctgtgggtcactgtctactatggagtgccagtttggaaggaagccaagacaactctgttttgcgccagcgacgccaaggcttatgacaaggaagtccacaacgtgtgggccacccacgcatgtgtcccaaccgaccccaacccacgcgaaatcgtgctggaaaacgtcacagaaaatttcaacatgtggaaaaacgatatggtggatcagatgcatgaggatattattagcctctgggaccagtctctgaagccatgtgtgaagttgacacctctctgtgtgacccttaactgtactaacgcccccgcctataacaactctatgcacggggagatgaaaaactgttccttcaacaccaccaccgaaatcagggacagaaaacagaaagcctatgccctgttctataagcccgatgtggtgccacttaaccgccgcgaagaaaataatggtactggcgaatatattctgattaactgtaacagctctacaattactcaggcttgccctaaagtcacctttgacccaatcccaatccactactgcgcccctgcaggatacgctatcctgaaatgcaataataagaccttcaacggaactggaccctgcaataacgtgtctacagtgcaatgtacccacggcattatgcccgtcgtctccacccaactgctgctcaatggcagcttggcagaagaggagatcattattaggagcgaaaacctcaccaacaatatcaagacaatcatcgtgcacctgaacaagtctgtggaaattgtgtgtaccaggcccaataacaacaccaggaagagcatccgcatcggacctggacaaactttctacgccaccggcgaaatcatcgggaacattagagaagcccactgcaacatctctaagagcaattggacatctacattggagcaagtgaaaaaaaagctgaaagagcactacaataagaccatcgagttcaaccctccttccggcggcgatctggaggtcacaacacactcctttaactgtaggggggagttcttttactgcaacacaacaaagctgtttagcaacaactccgacagcaataatgagactatcaccctgccttgcaagatcaagcaaatcattaacatgtggcagaaagtgggaagggcaatgtatgcacctcccatcgagggcaacatcacatgcaagtctaatatcaccggcctgttgctgactagagacggtggcaagaatactactaacgaaatcttcaggccaggtggagggaacatgaaagataattggcgctccgaactgtataagtacaaggtggtggagattgagcccctcggcgtcgcccccacaaagtctaagcgccgcgtggtggaaagagagaagagggctgtcggcctcggcgcagtgctgctggggttcttgggtgccgctgggtctacaatgggcgctgcctctattacactcaccgtgcaagctaggcagctgctgtccggtattgtgcaacaacagagcaatctcttgagagctatcgaggcccagcagcatatgctgcaacttacagtgtggggtattaagcagctgcaaactcgcgtcctggcaatcgaacgctacctgaaagaccagcaactcctgggtctgtggggctgctccggtaagatcatctgtaccacagccgtgccctggaacagcagctggtccaataagagccaagaggatatttgggataatatgacctggatgcaatgggatagagagatcagcaactacacaggaaccatttataggctcctggaagattctcagaaccagcaggagaagaacgagaaggacttgctcgccctggatagctggaaaaacctgtggaattggtttaacatcaccaactggctttggtacattaagattttcatcatgattgtgggaggcttgatcggcctgaggattatcttcggggtgcttgccattgtgaaaagggtcagacaaggatactccccattgtcctttcagaccttgactccaagcccacgcggacccgacaggttgggcaggatcgaggaggaaggaggcgaacaggataaggaccgctccatcagacttgttagcgggtttctggccctggcctgggatgatctgaggagcctgtgcctcttctcctatcaccacctccgcgatttcatcctcattgcagctagggctgctgagttgctgggacgctcctccctgagaggtctccagagaggctgggaggcactgaagtacctcgggaaccttgtgcaatacggcgggctggagctgaaaagatccgccatcaagctgttcgacaccatcgcaatcgccgttgcagagggcaccgacaggatcttggaggtcattcagaggatctgtcgcgccatccgccacatccccatcaggatcagacaaggattcgaggcagcactgcaatgatagttaattaaacgcgtggatcc

SEQ ID NO: 12 - protein, 852 aa, Human immunodeficiency virus type 1
KLMRVMGIQRNWPQWWIWGTLGFWMIIICRVVGNLNLWVTVYYGVPVWKEAKTTLFCASDAKAYDKEVHNVWATHACVPTDPNPREIVLENVTENFNMWKNDMVDQMHEDIISLWDQSLKPCVKLTPLCVTLNCTNAPAYNNSMHGEMKNCSFNTTTEIRDRKQKAYALFYKPDVVPLNRREENNGTGEYILINCNSSTITQACPKVTFDPIPIHYCAPAGYAILKCNNKTFNGTGPCNNVSTVQCTHGIMPVVSTQLLLNGSLAEEEIIIRSENLTNNIKTIIVHLNKSVEIVCTRPNNNTRKSIRIGPGQTFYATGEIIGNIREAHCNISKSNWTSTLEQVKKKLKEHYNKTIEFNPPSGGDLEVTTHSFNCRGEFFYCNTTKLFSNNSDSNNETITLPCKIKQIINMWQKVGRAMYAPPIEGNITCKSNITGLLLTRDGGKNTTNEIFRPGGGNMKDNWRSELYKYKVVEIEPLGVAPTKSKRRVVEREKRAVGLGAVLLGFLGAAGSTMGAASITLTVQARQLLSGIVQQQSNLLRAIEAQQHMLQLTVWGIKQLQTRVLAIERYLKDQQLLGLWGCSGKIICTTAVPWNSSWSNKSQEDIWDNMTWMQWDREISNYTGTIYRLLEDSQNQQEKNEKDLLALDSWKNLWNWFNITNWLWYIKIFIMIVGGLIGLRIIFGVLAIVKRVRQGYSPLSFQTLTPSPRGPDRLGRIEEEGGEQDKDRSIRLVSGFLALAWDDLRSLCLFSYHHLRDFILIAARAAELLGRSSLRGLQRGWEALKYLGNLVQYGGLELKRSAIKLFDTIAIAVAEGTDRILEVIQRICRAIRHIPIRIRQGFEAALQLIKRVD

SEQ ID NO: 13 - DNA, 2579 bp, Human immunodeficiency virus type 1
aggctaattttttagggaaaatttggccttcccacaaggggaggccagggaatttccttcagagcaggccaatgagagtgagggggatacagaggaattggccacaatggtggatatggggcatcttaggcttttggatgttaatgatttgtagtggggtgggaaacttgtgggtcacaatctattatggggtacctgtgtggagagaagcaaaaactactctattctgtgcatcagatgctaaagcatatgatagagaagtgcataatgtctgggctacacatgcctgtgtacccacagaccccaacccacaagaaatagttatgggaaatgtaacagaaaattttaacatgtggaaaaatgacatggtggatcagatgcatgaggatataatcaatttatgggatcaaagcctaaagccatgtgtaaagttaaccccactctgtgtcactttaaaatgtagtacctataatggtagtgataccaacgatatgagaaattgctctttcaatacaactacagaaataagggacaagaaacagacagtgtatgcacttttttataaacctgatatagtaccaattaatgagagtgagtatatattaatacattgcaatacctcaaccataacacaagcctgtccaaaggtctcttttgacccaattcctatacattattgtgctccagctggttatgcgattctaaagtgtaataataagacattcaatgggacgggaccatgccaaaatgtcagcacagtacaatgcacacatggaattaagccagtagtatcaactcaactactgttaaatggtagcatagcagaaggagagataataattagatctgaaaatctgacaaacaatgttaaaacaataatagtacaccttaatgaatctataggaattgtgtgtacaagacccggcaataatacaagaaaaagtataaggataggaccaggacaagcattctatacaaatcacataataggagatataagacaagcatattgtaacattagtaaacaagaatggaacaaaactttagaagaggtgagaaaaaaattgcaagaacacttcccaaataaaacaataaaatttaactcatcctcaggaggggacctagaaattacaacacatagctttaattgcagaggagaatttttctattgcaatacatcaaaactatttaatgatagtctagtaaatgatacagaaagtaattcaaccatcactattccatgcagaataaaacaaattataaacatgtggcaggaggtaggacgagcaatgtatgcccctcccattgcaggaaacataacatgtaaatcaaatatcacaggactactattgacacgtgatggaggaacagataacacaacagagatattcagacctggaggaggaaatatgaaggacaattggagaagtgaattatataaatataaagtagtagaaattaagccattgggaatagcacccactgaagcaaaaaggagagtggtggagagagaaaaaagagcagtgggaataggagctgtgctccttgggttcttgggagcagcaggaagcactatgggcgcggcgtcaataacgctgacggtacaggccagacaactgttgtctggtatagtgcaacagcaaagcaatttgctgagagctatagaggcgcaacagcatatgttgcaactcacagtctggggcattaagcagctccagacaagagtcctggctatagaaagatacctaaaggatcaacagctcctaggactttggggctgctctggaaaactcatctgcaccactaatgtgccttggaactccagttggagcaataaatctcaacaagctatttgggataacatgacatggatgcagtgggatagagaaattaataattacacaaacataatataccagttgcttgaggactcgcaaatccagcaggaacagaatgaaaaagatttattagcattggacaagtggcaaaatctgtggagttggtttagcataacaaattggctatggtatataaaaatattcataatgatagtaggaggcttaataggtttaagaataatttttgctgtgctatctatagtaaatagagttaggcagggatactcacctttgtcgtttcagacccttaccccaaacccgaggggacccgacaggctcggagaaatcgaagaagaaggtggagagcaagacagagacagatccgttcgattagtgagcggattcttaccacttgcctgggacgatctgcggagcctgtgcctcttcagctaccaccgattgagagacttcatattcgattgcagcgaggacagtggaacttctgggacgcagcagtctcaggggactccagaggggtgggaagtccttaaatatctgggaagccttgtgcagtattggggtctggagctaaaaagagtgctattagtctgcttgatacccatagcaatagcagtagctgaaggaacagataggattattgaattagtactaagattttgtagagctatccgcaacatacctacaagagtaagacagggctgtgaagcagctttgctataa

SEQ ID NO: 14 - protein, 858 aa, Human immunodeficiency virus type 1
ANFLGKIWPSHKGRPGNFLQSRPMRVRGIQRNWPQWWIWGILGFWMLMICSGVGNLWVTIYYGVPVWREAKTTLFCASDAKAYDREVHNVWATHACVPTDPNPQEIVMGNVTENFNMWKNDMVDQMHEDIINLWDQSLKPCVKLTPLCVTLKCSTYNGSDTNDMRNCSFNTTTEIRDKKQTVYALFYKPDIVPINESEYILIHCNTSTITQACPKVSFDPIPIHYCAPAGYAILKCNNKTFNGTGPCQNVSTVQCTHGIKPVVSTQLLLNGSIAEGEIIIRSENLTNNVKTIIVHLNESIGIVCTRPGNNTRKSIRIGPGQAFYTNHIIGDIRQAYCNISKQEWNKTLEEVRKKLQEHFPNKTIKFNSSSGGDLEITTHSFNCRGEFFYCNTSKLFNDSLVNDTESNSTITIPCRIKQIINMWQEVGRAMYAPPIAGNITCKSNITGLLLTRDGGTDNTTEIFRPGGGNMKDNWRSELYKYKVVEIKPLGIAPTEAKRRVVEREKRAVGIGAVLLGFLGAAGSTMGAASITLTVQARQLLSGIVQQQSNLLRAIEAQQHMLQLTVWGIKQLQTRVLAIERYLKDQQLLGLWGCSGKLICTTNVPWNSSWSNKSQQAIWDNMTWMQWDREINNYTNIIYQLLEDSQIQQEQNEKDLLALDKWQNLWSWFSITNWLWYIKIFIMIVGGLIGLRIIFAVLSIVNRVRQGYSPLSFQTLTPNPRGPDRLGEIEEEGGEQDRDRSVRLVSGFLPLAWDDLRSLCLFSYHRLRDFIFDCSEDSGTSGTQQSQGTPEGWEVLKYLGSLVQYWGLELKRVLLVCLIPIAIAVAEGTDRIIELVLRFCRAIRNIPTRVRQGCEAALL

SEQ ID NO: 15 - protein, 311 aa, Human immunodeficiency virus type 1
GEKLDKWEKIRLRPGGKKHYMLKHLVWASRELERFALNPGLLETSEGCKQIMKQLQPALQTGTEELRSLYNTVATLYCVHEKIEVRDTKEALDKIEEEQNKSQQCQQKTQQAKAADGGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTD

SEQ ID NO: 16 - protein, 277 aa, Human immunodeficiency virus type 1
LTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQSSMTKILEPFRAKNPEIVIYQYMDDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELE

SEQ ID NO: 17 - protein, 229 aa, Human immunodeficiency virus type 1
YCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEGKWNKTLQKVKKKLKEELYKYKVVEIKPLGIAPTEAKRRVVEREKRAVGIGAVFLGFLGAAGSTMGAASITLTVQARQLLSGIVQQQSNLLRAIEAQQHMLQLTVWGIKQL

SEQ ID NO: 18 - RNA, 1479 nt, Human immunodeficiency virus type 1
augggugcgagagcgucaauauuaagaggggaaaaauuagauaaaugggaaaaaauuagguuaaggccagggggaaagaaacauuauauguuaaaacacauaguaugggcaagcagggagcuggaaagauuugcacuuaacccuggccuuuuagaaacaucagaaggauguaaacaaauaaugaaacagcuacaaccagcucuccagacaggaacagaggaacuuaaaucauuauacaacacaguagcaacucucuauuguguacaugaaaagauagaaguacgagacaccaaggaagccuuagauaagauagaggaagaacaaaacaaaugucagcaaaaaacgcagcaggcaaaagcggcugacgggaaagucagucaaaauuauccuauagugcagaaucuccaagggcaaaugguacaucaagccauaucaccuagaaccuugaaugcauggguaaaaguaauagaagaaaaggcuuuuagcccagagguaauacccauguuuacagcauuaucagaaggagccaccccacaagauuuaaacaccauguuaaauacaguggggggacaucaagcagccaugcaaauguuaaaagauacuauuaaugaagaggcugcagaaugggauagaguacauccaguccaugcggggccuauugcaccaggccagaugagagaaccaaggggaagugacauagcaggaacuacuaguacccuucaggaacaaauagcauggaugacaaguaacccaccuauuccagugggagacaucuauaaaagauggauaauucugggguuaaauaaaauagugagaauguauagcccggucagcauuuuggacauaagacaagggccaaaggaacccuuucgagacuauguagaucgguucuuuaaaacuuuaagagcugaacaagcuacacaagaaguaaaaaauuggaugacagacaccuuguuaguccaaaaugcgaacccagauuguaagaccauuuugagagcauuaggaccaggggcuacauuagaagaaaugaugacagcaugucaaggggugggaggaccuggucacaaagcaagaguauuggcugaggcaaugagucaagcaaacaguggaaacauaaugaugcagagaagcaauuuuaaaggcccuagaagaauuguuaaauguuuuaacuguggcaaggaagggcacauagccagaaauugcagagccccuaggaaaaaaggcuguuggaaauguggaaaggaaggacaccaaaugaaagacuguacugaaaggcaggcuaauuuuuuagggaaaauuuggccuucccacaaggggaggccagggaauuuccuucagaacagaccagagccaacagccccaccagcagagagcuucagguucgaagagacaacccccgcuccgaaacaggagccgauagaaagggaacccuuaacuucccucaaaucacucuuuggcagcgaccccuugucucaauaa

SEQ ID NO: 19 - DNA, 1479 bp, Human immunodeficiency virus type 1
ttattgagacaaggggtcgctgccaaagagtgatttgagggaagttaagggttccctttctatcggctcctgtttcggagcgggggttgtctcttcgaacctgaagctctctgctggtggggctgttggctctggtctgttctgaaggaaattccctggcctccccttgtgggaaggccaaattttccctaaaaaattagcctgcctttcagtacagtctttcatttggtgtccttcctttccacatttccaacagccttttttcctaggggctctgcaatttctggctatgtgcccttccttgccacagttaaaacatttaacaattcttctagggcctttaaaattgcttctctgcatcattatgtttccactgtttgcttgactcattgcctcagccaatactcttgctttgtgaccaggtcctcccaccccttgacatgctgtcatcatttcttctaatgtagcccctggtcctaatgctctcaaaatggtcttacaatctgggttcgcattttggactaacaaggtgtctgtcatccaattttttacttcttgtgtagcttgttcagctcttaaagttttaaagaaccgatctacatagtctcgaaagggttcctttggcccttgtcttatgtccaaaatgctgaccgggctatacattctcactattttatttaaccccagaattatccatcttttatagatgtctcccactggaataggtgggttacttgtcatccatgctatttgttcctgaagggtactagtagttcctgctatgtcacttccccttggttctctcatctggcctggtgcaataggccccgcatggactggatgtactctatcccattctgcagcctcttcattaatagtatcttttaacatttgcatggctgcttgatgtccccccactgtatttaacatggtgtttaaatcttgtggggtggctccttctgataatgctgtaaacatgggtattacctctgggctaaaagccttttcttctattacttttacccatgcattcaaggttctaggtgatatggcttgatgtaccatttgcccttggagattctgcactataggataattttgactgactttcccgtcagccgcttttgcctgctgcgttttttgctgacatttgttttgttcttcctctatcttatctaaggcttccttggtgtctcgtacttctatcttttcatgtacacaatagagagttgctactgtgttgtataatgatttaagttcctctgttcctgtctggagagctggttgtagctgtttcattatttgtttacatccttctgatgtttctaaaaggccagggttaagtgcaaatctttccagctccctgcttgcccatactatgtgttttaacatataatgtttctttccccctggccttaacctaattttttcccatttatctaatttttcccctcttaatattgacgctctcgcacccat

SEQ ID NO: 20 - RNA, 1479 nt, Human immunodeficiency virus type 1
uuauugagacaaggggucgcugccaaagagugauuugagggaaguuaaggguucccuuucuaucggcuccuguuucggagcggggguugucucuucgaaccugaagcucucugcugguggggcuguuggcucuggucuguucugaaggaaauucccuggccuccccuugugggaaggccaaauuuucccuaaaaaauuagccugccuuucaguacagucuuucauuugguguccuuccuuuccacauuuccaacagccuuuuuuccuaggggcucugcaauuucuggcuaugugcccuuccuugccacaguuaaaacauuuaacaauucuucuagggccuuuaaaauugcuucucugcaucauuauguuuccacuguuugcuugacucauugccucagccaauacucuugcuuugugaccagguccucccaccccuugacaugcugucaucauuucuucuaauguagccccugguccuaaugcucucaaaauggucuuacaaucuggguucgcauuuuggacuaacaaggugucugucauccaauuuuuuacuucuuguguagcuuguucagcucuuaaaguuuuaaagaaccgaucuacauagucucgaaaggguuccuuuggcccuugucuuauguccaaaaugcugaccgggcuauacauucucacuauuuuauuuaaccccagaauuauccaucuuuuauagaugucucccacuggaauagguggguuacuugucauccaugcuauuuguuccugaaggguacuaguaguuccugcuaugucacuuccccuugguucucucaucuggccuggugcaauaggccccgcauggacuggauguacucuaucccauucugcagccucuucauuaauaguaucuuuuaacauuugcauggcugcuugauguccccccacuguauuuaacaugguguuuaaaucuugugggguggcuccuucugauaaugcuguaaacauggguauuaccucugggcuaaaagccuuuucuucuauuacuuuuacccaugcauucaagguucuaggugauauggcuugauguaccauuugcccuuggagauucugcacuauaggauaauuuugacugacuuucccgucagccgcuuuugccugcugcguuuuuugcugacauuuguuuuguucuuccucuaucuuaucuaaggcuuccuuggugucucguacuucuaucuuuucauguacacaauagagaguugcuacuguguuguauaaugauuuaaguuccucuguuccugucuggagagcugguuguagcuguuucauuauuuguuuacauccuucugauguuucuaaaaggccaggguuaagugcaaaucuuuccagcucccugcuugcccauacuauguguuuuaacauauaauguuucuuucccccuggccuuaaccuaauuuuuucccauuuaucuaauuuuuccccucuuaauauugacgcucucgcacccau

SEQ ID NO: 21 - RNA, 2997 nt, Human immunodeficiency virus type 1
uuuagggaaaauuuggccuucccacaaggggaggccagggaauuuccuucagaacagaccagagccaacagccccaccagcagagagcuucagguucgaagaaacaacccccgcuccgaaacaggagccgagagaaagggaacccuuaacuucccucaaaucacucuuuggcagcgaccccuugucucaauaaaaauagggggccagacaagggaggcucucuuagacacaggagcagaugauacaguauuagaagacauaaauuugccaggaaaauggaaaccaaaaaugauaggaggaauuggagguuuuaucaaaguaagacaguaugaucaaauacuuauagaaauuuguggaaaaaaggcuauagguacaguauuaguagggccuacaccugucaacauaauuggcagaaacauguugacucagcuuggaugcacacuaaacuuuccaaucagucccauugaaacuguaccaguaaaacugaagccaggaauggauggcccaaagguuaaacaauggccguuaacagaagagaaaauaaaagcauuaacagcaauuugugaagaaauggaaaaggaaggaaaaauuacaaaaauugggccugaaaauccauauaacacuccaauauuugccauaaaaaagaaagacagcacuaaguggagaaaauuaguagauuucagggaacucaauaaaagaacucaagacuuuugggagguucaauuaggaauaccacacccagcaggguuaaaaaagaaaaaaucagugacaguacuggaugugggagaugcauauuuuucaguuccuuuagaugaaggcuucaggaaauauacugcauucaccauaccuaguauaaacaaugaaacaccagggauuagauaucaauauaaugugcuuccacaaggauggaaagggucaccagcaauauuccaggguagcaugacaaaaaucuuagagcccuuuagagcucaaaauccagaaauagucaucuaucaauauauggaugacuuguauguaggaucugacuuagaaauagggcaacauagagcaaaaauagaagaguuaagagaacaucuauuaaaguggggauuuaccacaccagacaaaaaacaucagaaagaacccccauuucuuuggaugggguaugaacuccauccugacaaauggacaguacagccuauacagcugccagaaaaggauagcuggacugucaaugauauacagaaguuagugggaaaauuaaacugggcaagucagauuuacccagggauuaaaguaaggcaacuuuguaagcuccuuagggggaccaaagcacuaacagacauaguaccacuaacugaagaagcagaauuagaauuggcagagaacagggaaauucuaaaagaaccagugcauggaguauauuaugacccaucaaaagacuugauagcugaaauacagaaacagggggaugaccaauggacauaucaaauuuaccaagaaccauucaaaaaccugaagacaggaaaguaugcaaaaaggaggacuacccacacuaaugauguaaaacaguuaacagaggcagugcaaaaaauauccuuggaaagcauaguaauauggggaaagacuccuaaauuuagacuacccauccaaaaagaaacaugggaaauaugguggacagacuauuggcaagccacauggauuccugagugggaguuuguuaauaccccuccccuaguaaaacuaugguaccagcuagaaaaagaacccauagcaggagcagaaacuuucuauguagauggagcagcuaauagggaaacuaaaauaggaaaagcgggguauguuacugacagaggaaggcagaaaauuguaacucuaagugaaacaacaaaucagaagacugaauuacaagcaauucagcuagcuuugcaagauucagaaucagaaguaaacauaauaacagacucacaguacgcauuaggaaucauucaagcacaaccagauaggagugaaucagaguuggucaaucaaauaauagaacaauuaauaaaaaaggaaagggucuaucugucauggguaccagcacacaacggacuugcaggaaaugaacauguagauaaauuaguaaguaggggaaucaggaaagugcugguucuagauggaauagauaaggcucaugaagagcaugaaaaguaucacagcaauuggagagcaauggcuagugaguuuaaucugccacccguaguagcaagagaaauaguagccagcugugauaaaugucagcuaaaaggggaagccauacauggacaaguagauuguaguccggggauauggcaauuagauuguacacauuuagaaggaaaaaucauccugguagcaguccauguagccaguggcuacauagaagcagagguuaucccagcagaaacaggacaagaaacagcauacuauauacuaaaauuagcaggaagauggccagucaaaguaauacauacagacaauggcaguaauuucaccagugcugcaguuaaggcagccuguuggugggcagguauccaacaggaauuugggauucccuacaauccccaaagucagggaguaguagaauccaugaauaaagaauuaaagaaaaucauagggcagguaagagaucaagcugagcaccuuaagacagcaguacaaauggcaguauucauucacaauuuuaaaagaaaaggggggauugggggguacagugcaggggaaagaauaauagacauaauagcaacagacauacaaacuaaagaauuacaaaaacaaauuauaaaaauucaaaauuuucggguuuauuacagagacagcagagauccuauuuggaaaggaccagccaagcuacucuggaaaggugaaggggcaguaguaauacaagacaacagugacauaaagguaguaccaaggaggaaaguaaaaaucauuagggacuauggaaaacagauggcaggugcugauuguguggcagguagacaggaugaagauuag

SEQ ID NO: 22 - DNA, 2997 bp, Human immunodeficiency virus type 1
ctaatcttcatcctgtctacctgccacacaatcagcacctgccatctgttttccatagtccctaatgatttttactttcctccttggtactacctttatgtcactgttgtcttgtattactactgccccttcacctttccagagtagcttggctggtcctttccaaataggatctctgctgtctctgtaataaacccgaaaattttgaatttttataatttgtttttgtaattctttagtttgtatgtctgttgctattatgtctattattctttcccctgcactgtaccccccaatccccccttttcttttaaaattgtgaatgaatactgccatttgtactgctgtcttaaggtgctcagcttgatctcttacctgccctatgattttctttaattctttattcatggattctactactccctgactttggggattgtagggaatcccaaattcctgttggatacctgcccaccaacaggctgccttaactgcagcactggtgaaattactgccattgtctgtatgtattactttgactggccatcttcctgctaattttagtatatagtatgctgtttcttgtcctgtttctgctgggataacctctgcttctatgtagccactggctacatggactgctaccaggatgatttttccttctaaatgtgtacaatctaattgccatatccccggactacaatctacttgtccatgtatggcttccccttttagctgacatttatcacagctggctactatttctcttgctactacgggtggcagattaaactcactagccattgctctccaattgctgtgatacttttcatgctcttcatgagccttatctattccatctagaaccagcactttcctgattcccctacttactaatttatctacatgttcatttcctgcaagtccgttgtgtgctggtacccatgacagatagaccctttccttttttattaattgttctattatttgattgaccaactctgattcactcctatctggttgtgcttgaatgattcctaatgcgtactgtgagtctgttattatgtttacttctgattctgaatcttgcaaagctagctgaattgcttgtaattcagtcttctgatttgttgtttcacttagagttacaattttctgccttcctctgtcagtaacataccccgcttttcctattttagtttccctattagctgctccatctacatagaaagtttctgctcctgctatgggttctttttctagctggtaccatagttttactaggggaggggtattaacaaactcccactcaggaatccatgtggcttgccaatagtctgtccaccatatttcccatgtttctttttggatgggtagtctaaatttaggagtctttccccatattactatgctttccaaggatattttttgcactgcctctgttaactgttttacatcattagtgtgggtagtcctcctttttgcatactttcctgtcttcaggtttttgaatggttcttggtaaatttgatatgtccattggtcatccccctgtttctgtatttcagctatcaagtcttttgatgggtcataatatactccatgcactggttcttttagaatttccctgttctctgccaattctaattctgcttcttcagttagtggtactatgtctgttagtgctttggtccccctaaggagcttacaaagttgccttactttaatccctgggtaaatctgacttgcccagtttaattttcccactaacttctgtatatcattgacagtccagctatccttttctggcagctgtataggctgtactgtccatttgtcaggatggagttcataccccatccaaagaaatgggggttctttctgatgttttttgtctggtgtggtaaatccccactttaatagatgttctcttaactcttctatttttgctctatgttgccctatttctaagtcagatcctacatacaagtcatccatatattgatagatgactatttctggattttgagctctaaagggctctaagatttttgtcatgctaccctggaatattgctggtgaccctttccatccttgtggaagcacattatattgatatctaatccctggtgtttcattgtttatactaggtatggtgaatgcagtatatttcctgaagccttcatctaaaggaactgaaaaatatgcatctcccacatccagtactgtcactgattttttcttttttaaccctgctgggtgtggtattcctaattgaacctcccaaaagtcttgagttcttttattgagttccctgaaatctactaattttctccacttagtgctgtctttcttttttatggcaaatattggagtgttatatggattttcaggcccaatttttgtaatttttccttccttttccatttcttcacaaattgctgttaatgcttttattttctcttctgttaacggccattgtttaacctttgggccatccattcctggcttcagttttactggtacagtttcaatgggactgattggaaagtttagtgtgcatccaagctgagtcaacatgtttctgccaattatgttgacaggtgtaggccctactaatactgtacctatagccttttttccacaaatttctataagtatttgatcatactgtcttactttgataaaacctccaattcctcctatcatttttggtttccattttcctggcaaatttatgtcttctaatactgtatcatctgctcctgtgtctaagagagcctcccttgtctggccccctatttttattgagacaaggggtcgctgccaaagagtgatttgagggaagttaagggttccctttctctcggctcctgtttcggagcgggggttgtttcttcgaacctgaagctctctgctggtggggctgttggctctggtctgttctgaaggaaattccctggcctccccttgtgggaaggccaaattttccctaaa

SEQ ID NO: 23 - RNA, 2997 nt, Human immunodeficiency virus type 1
cuaaucuucauccugucuaccugccacacaaucagcaccugccaucuguuuuccauagucccuaaugauuuuuacuuuccuccuugguacuaccuuuaugucacuguugucuuguauuacuacugccccuucaccuuuccagaguagcuuggcugguccuuuccaaauaggaucucugcugucucuguaauaaacccgaaaauuuugaauuuuuauaauuuguuuuuguaauucuuuaguuuguaugucuguugcuauuaugucuauuauucuuuccccugcacuguaccccccaauccccccuuuucuuuuaaaauugugaaugaauacugccauuuguacugcugucuuaaggugcucagcuugaucucuuaccugcccuaugauuuucuuuaauucuuuauucauggauucuacuacucccugacuuuggggauuguagggaaucccaaauuccuguuggauaccugcccaccaacaggcugccuuaacugcagcacuggugaaauuacugccauugucuguauguauuacuuugacuggccaucuuccugcuaauuuuaguauauaguaugcuguuucuuguccuguuucugcugggauaaccucugcuucuauguagccacuggcuacauggacugcuaccaggaugauuuuuccuucuaaauguguacaaucuaauugccauauccccggacuacaaucuacuuguccauguauggcuuccccuuuuagcugacauuuaucacagcuggcuacuauuucucuugcuacuacggguggcagauuaaacucacuagccauugcucuccaauugcugugauacuuuucaugcucuucaugagccuuaucuauuccaucuagaaccagcacuuuccugauuccccuacuuacuaauuuaucuacauguucauuuccugcaaguccguugugugcugguacccaugacagauagacccuuuccuuuuuuauuaauuguucuauuauuugauugaccaacucugauucacuccuaucugguugugcuugaaugauuccuaaugcguacugugagucuguuauuauguuuacuucugauucugaaucuugcaaagcuagcugaauugcuuguaauucagucuucugauuuguuguuucacuuagaguuacaauuuucugccuuccucugucaguaacauaccccgcuuuuccuauuuuaguuucccuauuagcugcuccaucuacauagaaaguuucugcuccugcuauggguucuuuuucuagcugguaccauaguuuuacuaggggagggguauuaacaaacucccacucaggaauccauguggcuugccaauagucuguccaccauauuucccauguuucuuuuuggauggguagucuaaauuuaggagucuuuccccauauuacuaugcuuuccaaggauauuuuuugcacugccucuguuaacuguuuuacaucauuaguguggguaguccuccuuuuugcauacuuuccugucuucagguuuuugaaugguucuugguaaauuugauauguccauuggucaucccccuguuucuguauuucagcuaucaagucuuuugaugggucauaauauacuccaugcacugguucuuuuagaauuucccuguucucugccaauucuaauucugcuucuucaguuagugguacuaugucuguuagugcuuuggucccccuaaggagcuuacaaaguugccuuacuuuaaucccuggguaaaucugacuugcccaguuuaauuuucccacuaacuucuguauaucauugacaguccagcuauccuuuucuggcagcuguauaggcuguacuguccauuugucaggauggaguucauaccccauccaaagaaauggggguucuuucugauguuuuuugucuggugugguaaauccccacuuuaauagauguucucuuaacucuucuauuuuugcucuauguugcccuauuucuaagucagauccuacauacaagucauccauauauugauagaugacuauuucuggauuuugagcucuaaagggcucuaagauuuuugucaugcuacccuggaauauugcuggugacccuuuccauccuuguggaagcacauuauauugauaucuaaucccugguguuucauuguuuauacuagguauggugaaugcaguauauuuccugaagccuucaucuaaaggaacugaaaaauaugcaucucccacauccaguacugucacugauuuuuucuuuuuuaacccugcugggugugguauuccuaauugaaccucccaaaagucuugaguucuuuuauugaguucccugaaaucuacuaauuuucuccacuuagugcugucuuucuuuuuuauggcaaauauuggaguguuauauggauuuucaggcccaauuuuuguaauuuuuccuuccuuuuccauuucuucacaaauugcuguuaaugcuuuuauuuucucuucuguuaacggccauuguuuaaccuuugggccauccauuccuggcuucaguuuuacugguacaguuucaaugggacugauuggaaaguuuagugugcauccaagcugagucaacauguuucugccaauuauguugacagguguaggcccuacuaauacuguaccuauagccuuuuuuccacaaauuucuauaaguauuugaucauacugucuuacuuugauaaaaccuccaauuccuccuaucauuuuugguuuccauuuuccuggcaaauuuaugucuucuaauacuguaucaucugcuccugugucuaagagagccucccuugucuggcccccuauuuuuauugagacaaggggucgcugccaaagagugauuugagggaaguuaaggguucccuuucucucggcuccuguuucggagcggggguuguuucuucgaaccugaagcucucugcugguggggcuguuggcucuggucuguucugaaggaaauucccuggccuccccuugugggaaggccaaauuuucccuaaa

SEQ ID NO: 24 - RNA, 2535 nt, Human immunodeficiency virus type 1
augagagugauggggauacagaggaauuggccacaaugguggauauggggcaccuuaggcuuuuggaugauaauaauuuguagggugguggggaacuugaacuugugggucacagucuauuaugggguaccuguguggaaagaagcaaaaacuacucuauucugugcaucagaugcuaaagcauaugauaaagaaguacauaaugucugggcuacacaugccuguguacccacagaccccaacccacgagaaauaguuuuggaaaauguaacagaaaauuuuaacauguggaaaaaugacaugguggaucagaugcaugaggauauaaucaguuuaugggaucaaagccuaaaaccauguguaaaguugaccccacucugugucacuuuaaauuguacaaaugcaccugccuacaauaauagcaugcauggagaaaugaaaaauugcucuuucaauacaaccacagagauaagagauaggaaacagaaagcguaugcacuuuuuuauaaaccugauguagugccacuuaauaggagagaagagaauaaugggacaggagaguauauauuaauaaauugcaauuccucaaccauaacacaagccuguccaaaggucacuuuugacccaauuccuauacauuauugugcuccagcugguuaugcgauucuaaaguguaauaauaagacauucaaugggacaggaccaugcaauaaugucagcacaguacaauguacacauggaauuaugccagugguaucaacucaauuacuguuaaaugguagccuagcagaagaagagauaauaauuagaucugaaaaucugacaaacaauaucaaaacaauaauaguccaccuuaauaaaucuguagaaauuguguguacaagacccaacaauaauacaagaaaaaguauaaggauaggaccaggacaaacauucuaugcaacaggugaaauaauaggaaacauaagagaagcacauuguaacauuaguaaaaguaacuggaccaguacuuuagaacagguaaagaaaaaauuaaaagaacacuacaauaagacaauagaauuuaacccacccucaggaggggaucuagaaguuacaacacauagcuuuaauuguagaggagaauuuuucuauugcaauacaacaaaacuguuuucaaacaacagugauucaaacaacgaaaccaucacacucccaugcaagauaaaacaaauuauaaacauguggcagaagguaggacgagcaauguaugccccucccauugaaggaaacauaacauguaaaucaaauaucacaggacuacuauugacacgugauggaggaaagaauacaacaaaugagauauucagaccgggaggaggaaauaugaaggacaauuggagaagugaauuauauaaauauaaagugguagaaauugagccauugggaguagcacccacuaaaucaaaaaggagagugguggagagagaaaaaagagcagugggacuaggagcuguacuccuuggguucuugggagcagcaggaagcacuaugggcgcggcgucaauaacgcugacgguacaggccagacaacuguugucugguauagugcaacagcaaagcaauuugcugagagcuauagaggcgcaacagcauauguugcaacucacggucuggggcauuaagcagcuccagacaagagucuuggcuauagagagauaccuaaaggaucaacagcuccuagggcuuuggggcugcucuggaaaaaucaucugcaccacugcugugccuuggaacuccaguuggaguaauaaaucucaagaagauauuugggauaacaugaccuggaugcagugggauagagaaauuaguaauuacacaggcacaauauauagguuacuugaagacucgcaaaaccagcaggagaaaaaugaaaaagauuuauuagcauuggacaguuggaaaaacuuguggaauugguuuaacauaacaaauuggcugugguauauaaaaauauucaucaugauaguaggaggcuugauagguuugagaauaauuuuugguguacucgcuauagugaaaagaguuaggcagggauacucaccuuugucguuucagacccuuaccccaagcccgaggggucccgacaggcucggaagaaucgaagaagaagguggagagcaagacaaagacagauccauucgauuagugagcggauucuuagcacuugccugggacgaucugcggagccugugccucuucagcuaccaccacuugagagacuucauauugauugcagcgagagcagcggaacuucugggacgcagcagucucaggggacugcagagagggugggaagcccuuaaguaucugggaaaucuugugcaguaugggggucuggagcuaaaaagaagugcuauuaaacuguuugauaccauagcaauagcaguagcugaaggaacagauaggauucuugaaguaauacagagaauuuguagagcuauccgccacauaccuauaagaauaagacagggcuuugaagcagcuuugcaauaa

SEQ ID NO: 25 - DNA, 2535 bp, Human immunodeficiency virus type 1
ttattgcaaagctgcttcaaagccctgtcttattcttataggtatgtggcggatagctctacaaattctctgtattacttcaagaatcctatctgttccttcagctactgctattgctatggtatcaaacagtttaatagcacttctttttagctccagacccccatactgcacaagatttcccagatacttaagggcttcccaccctctctgcagtcccctgagactgctgcgtcccagaagttccgctgctctcgctgcaatcaatatgaagtctctcaagtggtggtagctgaagaggcacaggctccgcagatcgtcccaggcaagtgctaagaatccgctcactaatcgaatggatctgtctttgtcttgctctccaccttcttcttcgattcttccgagcctgtcgggacccctcgggcttggggtaagggtctgaaacgacaaaggtgagtatccctgcctaactcttttcactatagcgagtacaccaaaaattattctcaaacctatcaagcctcctactatcatgatgaatatttttatataccacagccaatttgttatgttaaaccaattccacaagtttttccaactgtccaatgctaataaatctttttcatttttctcctgctggttttgcgagtcttcaagtaacctatatattgtgcctgtgtaattactaatttctctatcccactgcatccaggtcatgttatcccaaatatcttcttgagatttattactccaactggagttccaaggcacagcagtggtgcagatgatttttccagagcagccccaaagccctaggagctgttgatcctttaggtatctctctatagccaagactcttgtctggagctgcttaatgccccagaccgtgagttgcaacatatgctgttgcgcctctatagctctcagcaaattgctttgctgttgcactataccagacaacagttgtctggcctgtaccgtcagcgttattgacgccgcgcccatagtgcttcctgctgctcccaagaacccaaggagtacagctcctagtcccactgctcttttttctctctccaccactctcctttttgatttagtgggtgctactcccaatggctcaatttctaccactttatatttatataattcacttctccaattgtccttcatatttcctcctcccggtctgaatatctcatttgttgtattctttcctccatcacgtgtcaatagtagtcctgtgatatttgatttacatgttatgtttccttcaatgggaggggcatacattgctcgtcctaccttctgccacatgtttataatttgttttatcttgcatgggagtgtgatggtttcgttgtttgaatcactgttgtttgaaaacagttttgttgtattgcaatagaaaaattctcctctacaattaaagctatgtgttgtaacttctagatcccctcctgagggtgggttaaattctattgtcttattgtagtgttcttttaattttttctttacctgttctaaagtactggtccagttacttttactaatgttacaatgtgcttctcttatgtttcctattatttcacctgttgcatagaatgtttgtcctggtcctatccttatactttttcttgtattattgttgggtcttgtacacacaatttctacagatttattaaggtggactattattgttttgatattgtttgtcagattttcagatctaattattatctcttcttctgctaggctaccatttaacagtaattgagttgataccactggcataattccatgtgtacattgtactgtgctgacattattgcatggtcctgtcccattgaatgtcttattattacactttagaatcgcataaccagctggagcacaataatgtataggaattgggtcaaaagtgacctttggacaggcttgtgttatggttgaggaattgcaatttattaatatatactctcctgtcccattattctcttctctcctattaagtggcactacatcaggtttataaaaaagtgcatacgctttctgtttcctatctcttatctctgtggttgtattgaaagagcaatttttcatttctccatgcatgctattattgtaggcaggtgcatttgtacaatttaaagtgacacagagtggggtcaactttacacatggttttaggctttgatcccataaactgattatatcctcatgcatctgatccaccatgtcatttttccacatgttaaaattttctgttacattttccaaaactatttctcgtgggttggggtctgtgggtacacaggcatgtgtagcccagacattatgtacttctttatcatatgctttagcatctgatgcacagaatagagtagtttttgcttctttccacacaggtaccccataatagactgtgacccacaagttcaagttccccaccaccctacaaattattatcatccaaaagcctaaggtgccccatatccaccattgtggccaattcctctgtatccccatcactctcat

SEQ ID NO: 26 - RNA, 2535 nt, Human immunodeficiency virus type 1
uuauugcaaagcugcuucaaagcccugucuuauucuuauagguauguggcggauagcucuacaaauucucuguauuacuucaagaauccuaucuguuccuucagcuacugcuauugcuaugguaucaaacaguuuaauagcacuucuuuuuagcuccagacccccauacugcacaagauuucccagauacuuaagggcuucccacccucucugcaguccccugagacugcugcgucccagaaguuccgcugcucucgcugcaaucaauaugaagucucucaaguggugguagcugaagaggcacaggcuccgcagaucgucccaggcaagugcuaagaauccgcucacuaaucgaauggaucugucuuugucuugcucuccaccuucuucuucgauucuuccgagccugucgggaccccucgggcuugggguaagggucugaaacgacaaaggugaguaucccugccuaacucuuuucacuauagcgaguacaccaaaaauuauucucaaaccuaucaagccuccuacuaucaugaugaauauuuuuauauaccacagccaauuuguuauguuaaaccaauuccacaaguuuuuccaacuguccaaugcuaauaaaucuuuuucauuuuucuccugcugguuuugcgagucuucaaguaaccuauauauugugccuguguaauuacuaauuucucuaucccacugcauccaggucauguuaucccaaauaucuucuugagauuuauuacuccaacuggaguuccaaggcacagcaguggugcagaugauuuuuccagagcagccccaaagcccuaggagcuguugauccuuuagguaucucucuauagccaagacucuugucuggagcugcuuaaugccccagaccgugaguugcaacauaugcuguugcgccucuauagcucucagcaaauugcuuugcuguugcacuauaccagacaacaguugucuggccuguaccgucagcguuauugacgccgcgcccauagugcuuccugcugcucccaagaacccaaggaguacagcuccuagucccacugcucuuuuuucucucuccaccacucuccuuuuugauuuagugggugcuacucccaauggcucaauuucuaccacuuuauauuuauauaauucacuucuccaauuguccuucauauuuccuccucccggucugaauaucucauuuguuguauucuuuccuccaucacgugucaauaguaguccugugauauuugauuuacauguuauguuuccuucaaugggaggggcauacauugcucguccuaccuucugccacauguuuauaauuuguuuuaucuugcaugggagugugaugguuucguuguuugaaucacuguuguuugaaaacaguuuuguuguauugcaauagaaaaauucuccucuacaauuaaagcuauguguuguaacuucuagauccccuccugaggguggguuaaauucuauugucuuauuguaguguucuuuuaauuuuuucuuuaccuguucuaaaguacugguccaguuacuuuuacuaauguuacaaugugcuucucuuauguuuccuauuauuucaccuguugcauagaauguuuguccugguccuauccuuauacuuuuucuuguauuauuguugggucuuguacacacaauuucuacagauuuauuaagguggacuauuauuguuuugauauuguuugucagauuuucagaucuaauuauuaucucuucuucugcuaggcuaccauuuaacaguaauugaguugauaccacuggcauaauuccauguguacauuguacugugcugacauuauugcaugguccugucccauugaaugucuuauuauuacacuuuagaaucgcauaaccagcuggagcacaauaauguauaggaauugggucaaaagugaccuuuggacaggcuuguguuaugguugaggaauugcaauuuauuaauauauacucuccugucccauuauucucuucucuccuauuaaguggcacuacaucagguuuauaaaaaagugcauacgcuuucuguuuccuaucucuuaucucugugguuguauugaaagagcaauuuuucauuucuccaugcaugcuauuauuguaggcaggugcauuuguacaauuuaaagugacacagaguggggucaacuuuacacaugguuuuaggcuuugaucccauaaacugauuauauccucaugcaucugauccaccaugucauuuuuccacauguuaaaauuuucuguuacauuuuccaaaacuauuucucguggguuggggucuguggguacacaggcauguguagcccagacauuauguacuucuuuaucauaugcuuuagcaucugaugcacagaauagaguaguuuuugcuucuuuccacacagguaccccauaauagacugugacccacaaguucaaguuccccaccacccuacaaauuauuaucauccaaaagccuaaggugccccauauccaccauuguggccaauuccucuguauccccaucacucucau

SEQ ID NO: 27 - RNA, 2579 nt, Human immunodeficiency virus type 1
aggcuaauuuuuuagggaaaauuuggccuucccacaaggggaggccagggaauuuccuucagagcaggccaaugagagugagggggauacagaggaauuggccacaaugguggauauggggcaucuuaggcuuuuggauguuaaugauuuguaguggggugggaaacuugugggucacaaucuauuaugggguaccuguguggagagaagcaaaaacuacucuauucugugcaucagaugcuaaagcauaugauagagaagugcauaaugucugggcuacacaugccuguguacccacagaccccaacccacaagaaauaguuaugggaaauguaacagaaaauuuuaacauguggaaaaaugacaugguggaucagaugcaugaggauauaaucaauuuaugggaucaaagccuaaagccauguguaaaguuaaccccacucugugucacuuuaaaauguaguaccuauaaugguagugauaccaacgauaugagaaauugcucuuucaauacaacuacagaaauaagggacaagaaacagacaguguaugcacuuuuuuauaaaccugauauaguaccaauuaaugagagugaguauauauuaauacauugcaauaccucaaccauaacacaagccuguccaaaggucucuuuugacccaauuccuauacauuauugugcuccagcugguuaugcgauucuaaaguguaauaauaagacauucaaugggacgggaccaugccaaaaugucagcacaguacaaugcacacauggaauuaagccaguaguaucaacucaacuacuguuaaaugguagcauagcagaaggagagauaauaauuagaucugaaaaucugacaaacaauguuaaaacaauaauaguacaccuuaaugaaucuauaggaauuguguguacaagacccggcaauaauacaagaaaaaguauaaggauaggaccaggacaagcauucuauacaaaucacauaauaggagauauaagacaagcauauuguaacauuaguaaacaagaauggaacaaaacuuuagaagaggugagaaaaaaauugcaagaacacuucccaaauaaaacaauaaaauuuaacucauccucaggaggggaccuagaaauuacaacacauagcuuuaauugcagaggagaauuuuucuauugcaauacaucaaaacuauuuaaugauagucuaguaaaugauacagaaaguaauucaaccaucacuauuccaugcagaauaaaacaaauuauaaacauguggcaggagguaggacgagcaauguaugccccucccauugcaggaaacauaacauguaaaucaaauaucacaggacuacuauugacacgugauggaggaacagauaacacaacagagauauucagaccuggaggaggaaauaugaaggacaauuggagaagugaauuauauaaauauaaaguaguagaaauuaagccauugggaauagcacccacugaagcaaaaaggagagugguggagagagaaaaaagagcagugggaauaggagcugugcuccuuggguucuugggagcagcaggaagcacuaugggcgcggcgucaauaacgcugacgguacaggccagacaacuguugucugguauagugcaacagcaaagcaauuugcugagagcuauagaggcgcaacagcauauguugcaacucacagucuggggcauuaagcagcuccagacaagaguccuggcuauagaaagauaccuaaaggaucaacagcuccuaggacuuuggggcugcucuggaaaacucaucugcaccacuaaugugccuuggaacuccaguuggagcaauaaaucucaacaagcuauuugggauaacaugacauggaugcagugggauagagaaauuaauaauuacacaaacauaauauaccaguugcuugaggacucgcaaauccagcaggaacagaaugaaaaagauuuauuagcauuggacaaguggcaaaaucuguggaguugguuuagcauaacaaauuggcuaugguauauaaaaauauucauaaugauaguaggaggcuuaauagguuuaagaauaauuuuugcugugcuaucuauaguaaauagaguuaggcagggauacucaccuuugucguuucagacccuuaccccaaacccgaggggacccgacaggcucggagaaaucgaagaagaagguggagagcaagacagagacagauccguucgauuagugagcggauucuuaccacuugccugggacgaucugcggagccugugccucuucagcuaccaccgauugagagacuucauauucgauugcagcgaggacaguggaacuucugggacgcagcagucucaggggacuccagaggggugggaaguccuuaaauaucugggaagccuugugcaguauuggggucuggagcuaaaaagagugcuauuagucugcuugauacccauagcaauagcaguagcugaaggaacagauaggauuauugaauuaguacuaagauuuuguagagcuauccgcaacauaccuacaagaguaagacagggcugugaagcagcuuugcuauaa

SEQ ID NO: 28 - DNA, 2579 bp, Human immunodeficiency virus type 1
ttatagcaaagctgcttcacagccctgtcttactcttgtaggtatgttgcggatagctctacaaaatcttagtactaattcaataatcctatctgttccttcagctactgctattgctatgggtatcaagcagactaatagcactctttttagctccagaccccaatactgcacaaggcttcccagatatttaaggacttcccacccctctggagtcccctgagactgctgcgtcccagaagttccactgtcctcgctgcaatcgaatatgaagtctctcaatcggtggtagctgaagaggcacaggctccgcagatcgtcccaggcaagtggtaagaatccgctcactaatcgaacggatctgtctctgtcttgctctccaccttcttcttcgatttctccgagcctgtcgggtcccctcgggtttggggtaagggtctgaaacgacaaaggtgagtatccctgcctaactctatttactatagatagcacagcaaaaattattcttaaacctattaagcctcctactatcattatgaatatttttatataccatagccaatttgttatgctaaaccaactccacagattttgccacttgtccaatgctaataaatctttttcattctgttcctgctggatttgcgagtcctcaagcaactggtatattatgtttgtgtaattattaatttctctatcccactgcatccatgtcatgttatcccaaatagcttgttgagatttattgctccaactggagttccaaggcacattagtggtgcagatgagttttccagagcagccccaaagtcctaggagctgttgatcctttaggtatctttctatagccaggactcttgtctggagctgcttaatgccccagactgtgagttgcaacatatgctgttgcgcctctatagctctcagcaaattgctttgctgttgcactataccagacaacagttgtctggcctgtaccgtcagcgttattgacgccgcgcccatagtgcttcctgctgctcccaagaacccaaggagcacagctcctattcccactgctcttttttctctctccaccactctcctttttgcttcagtgggtgctattcccaatggcttaatttctactactttatatttatataattcacttctccaattgtccttcatatttcctcctccaggtctgaatatctctgttgtgttatctgttcctccatcacgtgtcaatagtagtcctgtgatatttgatttacatgttatgtttcctgcaatgggaggggcatacattgctcgtcctacctcctgccacatgtttataatttgttttattctgcatggaatagtgatggttgaattactttctgtatcatttactagactatcattaaatagttttgatgtattgcaatagaaaaattctcctctgcaattaaagctatgtgttgtaatttctaggtcccctcctgaggatgagttaaattttattgttttatttgggaagtgttcttgcaatttttttctcacctcttctaaagttttgttccattcttgtttactaatgttacaatatgcttgtcttatatctcctattatgtgatttgtatagaatgcttgtcctggtcctatccttatactttttcttgtattattgccgggtcttgtacacacaattcctatagattcattaaggtgtactattattgttttaacattgtttgtcagattttcagatctaattattatctctccttctgctatgctaccatttaacagtagttgagttgatactactggcttaattccatgtgtgcattgtactgtgctgacattttggcatggtcccgtcccattgaatgtcttattattacactttagaatcgcataaccagctggagcacaataatgtataggaattgggtcaaaagagacctttggacaggcttgtgttatggttgaggtattgcaatgtattaatatatactcactctcattaattggtactatatcaggtttataaaaaagtgcatacactgtctgtttcttgtcccttatttctgtagttgtattgaaagagcaatttctcatatcgttggtatcactaccattataggtactacattttaaagtgacacagagtggggttaactttacacatggctttaggctttgatcccataaattgattatatcctcatgcatctgatccaccatgtcatttttccacatgttaaaattttctgttacatttcccataactatttcttgtgggttggggtctgtgggtacacaggcatgtgtagcccagacattatgcacttctctatcatatgctttagcatctgatgcacagaatagagtagtttttgcttctctccacacaggtaccccataatagattgtgacccacaagtttcccaccccactacaaatcattaacatccaaaagcctaagatgccccatatccaccattgtggccaattcctctgtatccccctcactctcattggcctgctctgaaggaaattccctggcctccccttgtgggaaggccaaattttccctaaaaaattagcct

SEQ ID NO: 29 - RNA, 2579 nt, Human immunodeficiency virus type 1
uuauagcaaagcugcuucacagcccugucuuacucuuguagguauguugcggauagcucuacaaaaucuuaguacuaauucaauaauccuaucuguuccuucagcuacugcuauugcuauggguaucaagcagacuaauagcacucuuuuuagcuccagaccccaauacugcacaaggcuucccagauauuuaaggacuucccaccccucuggaguccccugagacugcugcgucccagaaguuccacuguccucgcugcaaucgaauaugaagucucucaaucggugguagcugaagaggcacaggcuccgcagaucgucccaggcaagugguaagaauccgcucacuaaucgaacggaucugucucugucuugcucuccaccuucuucuucgauuucuccgagccugucggguccccucggguuugggguaagggucugaaacgacaaaggugaguaucccugccuaacucuauuuacuauagauagcacagcaaaaauuauucuuaaaccuauuaagccuccuacuaucauuaugaauauuuuuauauaccauagccaauuuguuaugcuaaaccaacuccacagauuuugccacuuguccaaugcuaauaaaucuuuuucauucuguuccugcuggauuugcgaguccucaagcaacugguauauuauguuuguguaauuauuaauuucucuaucccacugcauccaugucauguuaucccaaauagcuuguugagauuuauugcuccaacuggaguuccaaggcacauuaguggugcagaugaguuuuccagagcagccccaaaguccuaggagcuguugauccuuuagguaucuuucuauagccaggacucuugucuggagcugcuuaaugccccagacugugaguugcaacauaugcuguugcgccucuauagcucucagcaaauugcuuugcuguugcacuauaccagacaacaguugucuggccuguaccgucagcguuauugacgccgcgcccauagugcuuccugcugcucccaagaacccaaggagcacagcuccuauucccacugcucuuuuuucucucuccaccacucuccuuuuugcuucagugggugcuauucccaauggcuuaauuucuacuacuuuauauuuauauaauucacuucuccaauuguccuucauauuuccuccuccaggucugaauaucucuguuguguuaucuguuccuccaucacgugucaauaguaguccugugauauuugauuuacauguuauguuuccugcaaugggaggggcauacauugcucguccuaccuccugccacauguuuauaauuuguuuuauucugcauggaauagugaugguugaauuacuuucuguaucauuuacuagacuaucauuaaauaguuuugauguauugcaauagaaaaauucuccucugcaauuaaagcuauguguuguaauuucuagguccccuccugaggaugaguuaaauuuuauuguuuuauuugggaaguguucuugcaauuuuuuucucaccucuucuaaaguuuuguuccauucuuguuuacuaauguuacaauaugcuugucuuauaucuccuauuaugugauuuguauagaaugcuuguccugguccuauccuuauacuuuuucuuguauuauugccgggucuuguacacacaauuccuauagauucauuaagguguacuauuauuguuuuaacauuguuugucagauuuucagaucuaauuauuaucucuccuucugcuaugcuaccauuuaacaguaguugaguugauacuacuggcuuaauuccaugugugcauuguacugugcugacauuuuggcauggucccgucccauugaaugucuuauuauuacacuuuagaaucgcauaaccagcuggagcacaauaauguauaggaauugggucaaaagagaccuuuggacaggcuuguguuaugguugagguauugcaauguauuaauauauacucacucucauuaauugguacuauaucagguuuauaaaaaagugcauacacugucuguuucuugucccuuauuucuguaguuguauugaaagagcaauuucucauaucguugguaucacuaccauuauagguacuacauuuuaaagugacacagagugggguuaacuuuacacauggcuuuaggcuuugaucccauaaauugauuauauccucaugcaucugauccaccaugucauuuuuccacauguuaaaauuuucuguuacauuucccauaacuauuucuuguggguuggggucuguggguacacaggcauguguagcccagacauuaugcacuucucuaucauaugcuuuagcaucugaugcacagaauagaguaguuuuugcuucucuccacacagguaccccauaauagauugugacccacaaguuucccaccccacuacaaaucauuaacauccaaaagccuaagaugccccauauccaccauuguggccaauuccucuguaucccccucacucucauuggccugcucugaaggaaauucccuggccuccccuugugggaaggccaaauuuucccuaaaaaauuagccu

SEQ ID NO: 30 - protein, 307 aa, Human immunodeficiency virus type 1
GEKLDTWEKIRLRPGGKKHYMLKHIVWASRELERFALNPGLLETSEGCKQIMKQLQPALQTGTEELKSLYNTVATLYCVHEKIEVRDTKEALDKIEEEQNKCQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRVHPVHAGPIAPGQMREPRGSDIAGTTSTLQEQIAWMTSNPPIPVGDIYKRWIILGLNKIVRMYSPVSILDIRQGPKEPFRDYVDRFFKTLRAEQATQEVKNWMTD

SEQ ID NO: 31 - protein, 278 aa, Human immundeficiency virus type 1
PLTEEKIKALTAICEEMEKEGKITKIGPENPYNTPIFAIKKKDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQGSMTKILEPFRAQNPEIVIYQYMDDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGTKALTDIVPLTEEAELE

SEQ ID NO: 32 - protein, 335 aa, Human immunodeficiency virus type 1
YCAPAGYAILKCNNKTFNGTGPCNNVSTVQCTHGIMPVVSTQLLLNGSLAEEEIIIRSENLTNNIKTIIVHLNKSVEIVCTRPNNNTRKSIRIGPGQTFYATGEIIGNIREAHCNISKSNWTSTLEQVKKKLKEHYNKTIEFNPPSGGDLEVTTHSFNCRGEFFYCNTTKLFSNNSDSNNETITLPCKIKQIINMWQKVGRAMYAPPIEGNITCKSNITGLLLTRDGGKNTTNEIFRPGGGNMKDNWRSELYKYKVVEIEPLGVAPTKSKRRVVEREKRAVGLGAVLLGFLGAAGSTMGAASITLTVQARQLLSGIVQQQSNLLRAIEAQQHMLQ

(19) United States
(12) Patent Application Publication
(10) Pub. No.: US 2004/0116660 A1
(43) Pub. Date: Jun. 17, 2004

(54) Process for the selection of hiv-1 subtype c Isolates, selected hiv-1 subtype c isolates, their genes and modifications and derivatives thereof

(76) Inventors: Robert Edward Johnston, Chapel Hill, NC (US); Salim Abdool Karim, Cape Town (ZA); Lynn Morris, Sandringham (ZA); Ronald Ivar Swanstrom, Chapel Hill, NC (US); Carolynn Williamson, Cape Town (ZA)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/332,413

(22) PCT Filed: Jul. 9, 2001
(86) PCT No.: PCT/IB01/01208

(30) Foreign Application Priority Data
Jul. 10, 2000 (ZA) 2000/3437
Sep. 15, 2000 (ZA) 2000/4924

(51) Int. Cl. C12Q 1/70; C12N 7/00; C12N 15/00; C12N 15/63; C07H 21/04; C12N 15/09; C12N 15/74; C07K 14/00; C07K 17/00; C07K 1/00; C12N 15/70; C12N 7/01

(52) U.S. Cl. 530/350; 536/23.72; 435/5; 530/826; 435/235.1; 435/320.1

(57) ABSTRACT

The invention provides a process for the selection of HIV-1 subtype (clade) C isolates, selected HIV-1 subtype C isolates, their genes and modifications and derivatives thereof for use in prophylactic and therapeutic vaccines to produce proteins and polypeptides for the purpose of eliciting protection against HIV infection or disease. The process for the selection of HIV subtype isolates comprises the steps of isolating viruses from recently infected subjects; generating a consensus sequence for at least part of at least one HIV gene by identifying the most common codon or amino acid among the isolated viruses; and selecting the isolated virus or viruses with a high sequence identity to the consensus sequence. HIV-1 subtype C isolates, designated Du422, Du 151 and Du 179 (assigned Accession Numbers 01032114, 00072724 and 00072725, respectively, by the European Collection of Cell Cultures) are also provided.

### BACKGROUND TO THE INVENTION

THIS invention relates to a process for the selection of HIV-1 subtype (lade) C isolates, selected HIV-1 subtype C isolates, their genes and modifications and derivatives thereof for use in prophylactic and therapeutic vaccines to produce proteins and polypeptides for the purpose of eliciting protection against HIV infection or disease.

The disease acquired immunodeficiency syndrome (AIDS) is caused by human immunodeficiency virus (HIV). Over 34 million people worldwide are thought to be living with HIV/AIDS, with over 90% of infected people living in developing countries (UNAIDS, 1999). It is estimated that 24 million infected people reside in sub-Saharan Africa and that South Africa currently has one of the world's fastest growing HIV-1 epidemics. At the end of 1999, over 22% of pregnant women attending government antenatal clinics in South Africa were HIV positive (Department of Health, 2000). A preventative vaccine is considered to be the only feasible way to control this epidemic in the long term.

HIV shows remarkable genetic diversity that has confounded the development of a vaccine. The molecular basis of variation resides in the viral enzyme reverse transcriptase which not only introduces an error every round of replication, but also promotes recombination between viral RNAs. Based on phylogenetic analysis of sequences, HIV has been classified into a number of groups: the M (major group) which comprises subtypes A to H and K, the O (outlier group) and the N (non-M, non-O group). Recently recombinant viruses have been more frequently identified and there are a number which have spread significantly and established epidemics (circulating recombinant forms or CRF) such as subtype A/G recombinant in West Africa, and CRF A/E recombinant in Thailand (Robertson et al., 2000).

Subtype C predominates in the Southern African region which includes Botswana, Zimbabwe, Zambia, Malawi, Mozambique and South Africa. In addition, increasing numbers of subtype C infections are being detected in the Southern region of Tanzania. This subtype also predominates in Ethiopia and India and is becoming more important in China.

A possible further obstacle to vaccine development is that the biological properties of HIV change as disease progresses. HIV requires two receptors to infect cells, the CD4 and co-receptors of which CCR5 and CXCR4 are the major co-receptors used by HIV-1 strains. The most commonly transmitted phenotype is non-syncytium inducing (NSI), macrophage-tropic viruses that utilise the CCR5 co-receptor for entry (R5 viruses). Langerhans cells in the mucosa are thought to selectively pick up R5 variants at the portal of entry and transport them to the lymph nodes where they undergo replication and expansion. As the infection progresses, viruses evolve that have increased replicative capacity and the ability to grow in T cell lines. These syncytium-inducing (SI) T-tropic viruses use CXCR4 in conjunction with or in preference to CCR5, and in some cases also use other minor co-receptors (Connor et al., 1997, Richman & Bozzette, 1994). However HIV-1 subtype C viruses appear to be unusual in that they do not readily undergo this phenotypic switch, as R5 viruses are also predominant in patients with advanced AIDS (Bjorndal et al., 1999, Peeters et al., 1999, Ping et al., 1999, Tscherning et al., 1998, Scarlatti et al., 1997).

### SUMMARY OF THE INVENTION

According to one aspect of the invention a process for the selection of HIV subtype isolates for use in the development of prophylactic and therapeutic pharmaceutical composition comprises the following steps:

isolating viruses from recently infected subjects;

generating a consensus sequence for at least part of at least one HIV gene by identifying the most common codon or amino acid among the isolated viruses at each position along at least part of the gene; and

selecting the isolated virus or viruses with a high sequence identity to the consensus sequence, a phenotype which is associated with transmission for the particular HIV subtype.

The isolated virus may be of the same subtype as a likely challenge strain.

The HIV subtype is preferably HIV-1 subtype C.

For HIV-1 subtype C, the phenotype which is associated with transmission is typically a virus that utilises the CCR5 co-receptor and is non syncitium inducing (NSI).

According to another aspect of the invention an HIV-1 subtype C isolate, designated Du422 and assigned Provisional Accession Number 01032114 by the European Collection of Cell Cultures, is provided.

According to another aspect of the invention an HIV-1 subtype C isolate, designated Du151 and assigned Accession Number 00072724 by the European Collection of Cell Cultures, is provided.

According to another aspect of the invention an HIV-1 subtype C isolate, designated Du179 and assigned Accession Number 00072725 by the European Collection of Cell Cultures, is provided.

According to another aspect of the invention a molecule is provided, the molecule having:

(i) the nucleotide sequence set out in sequence as set out in Sequence I.D. No. 1;

(ii) an RNA sequence corresponding to the nucleotide sequence set out in Sequence I.D. No. 1;

(iii) a sequence which will hybridise to the nucleotide sequence set out in Sequence I.D. No. 1 or an RNA sequence corresponding to it, under strict hybridisation conditions;

(iv) a sequence which is homologous to the nucleotide sequence set out in Sequence I.D. No. 1 or an RNA sequence corresponding to it; or

(v) a sequence which is a modification or derivative of the sequence of any one of (i) to (iv).

The modified sequence is preferably that set out in Sequence I.D. No. 7.

According to another aspect of the invention a molecule is provided, the molecule having:

(i) the nucleotide sequence set out in Sequence I.D. No. 3;

(ii) an RNA sequence corresponding to the nucleotide sequence set out in Sequence I.D. No. 3;

(iii) a sequence which will hybridise to the nucleotide sequence set out in Sequence I.D. No. 3 or an RNA sequence corresponding to it, under strict hybridisation conditions;

(iv) a sequence which is homologous to the nucleotide sequence set out in Sequence I.D. No. 3 or an RNA sequence corresponding to it; or

(v) a sequence which is a modification or derivative of the sequence of any one of (i) to (iv).

The modified sequence is preferably that set out in Sequence I.D. No. 9.

According to another aspect of the invention a molecule is provided, the molecule having:

(i) the nucleotide sequence set out in Sequence I.D. No. 5;

(ii) an RNA sequence corresponding to the nucleotide sequence set out in Sequence I.D. No. 5;

(iii) a sequence which will hybridise to the nucleotide sequence set out in Sequence I.D. No. 5 or an RNA sequence corresponding to it, under strict hybridisation conditions;

(iv). a sequence which is homologous to the nucleotide sequence set out in Sequence I.D. No. 5 or an RNA sequence corresponding to it; or

(v) a sequence which is a modification or derivative of the sequence of any one of (i) to (iv).

The modified sequence is preferably that set out in Sequence I.D. No. 11.

According to another aspect of the invention a molecule is provided, the molecule having:

(i) the nucleotide sequence set out in Sequence I.D. No. 13;

(ii) an RNA sequence corresponding to the nucleotide sequence set out in Sequence I.D. No. 13;

(iii) a sequence which will hybridise to the nucleotide sequence set out in Sequence I.D. No. 13 or an RNA sequence corresponding to it, under strict hybridisation conditions;

(iv) a sequence which is homologous to the nucleotide sequence set out in Sequence I.D. No. 13 or an RNA sequence corresponding to it; or

(v) a sequence which is a modification or derivative of the sequence of any one of (i) to (iv).

The modified sequence preferably has similar or the same modifications as those set out in Sequence I.D. No. 11 for the env gene of the isolate Du151.

According to another aspect of the invention a polypeptide is provided, the polypeptide having:

(i) the amino acid sequence set out in Sequence I.D. No. 2; or

(ii) a sequence which is a modification or derivative of the amino acid sequence set out in Sequence I.D. No. 2.

The modified sequence is preferably that set out in Sequence I.D. No. 8.

According to another aspect of the invention a polypeptide is provided, the polypeptide having:

(i) the amino acid sequence set out in Sequence I.D. No. 4; or

(ii) a sequence which is a modification or derivative of the amino acid sequence set out in Sequence I.D. No. 4.

The modified sequence is preferably that set out in Sequence I.D. No. 10.

According to another aspect of the invention a polypeptide is provided, the polypeptide having:

(i) the amino acid sequence set out in Sequence I.D. No. 6; or

(ii) a sequence which is a modification or derivative of the amino acid sequence set out in Sequence I.D. No. 6.

The modified sequence is preferably that set out in Sequence I.D. No. 12.

According to another aspect of the invention a polypeptide is provided, the polypeptide having:

(i) the amino acid sequence set out in Sequence I.D. No. 14;

(ii) a sequence which is a modification or derivative of the amino acid sequence set out in Sequence I.D. No. 14.

The modified sequence preferably has similar or the same modifications as those set out in Sequence I.D. No. 12 for the amino acid sequence of the env gene of the isolate Du151.

According to another aspect of the invention a consensus amino acid sequence for the partial gag gene of HIV-1 subtype C is the following:

GEKLDKWEKI RLRPGGKKHY MLKHLVWASR ELERFALNPG

LLETSEGCKQ⁵⁰

IMKQLQPALQ TGTEELRSLY NTVATLYCVH EKIEVRDTKE

ALDKIEEEQN¹⁰⁰

KDQQ-CQQKT QQAKAADGG- KVSQNYPIVQ NLQGQMVHQA

ISPRTLNAWV¹⁵⁰

EEKAFSP EVIPMFTALS EGATPQDLNT MLNTVGGHQA

AMQMLKDTIN²⁰⁰

EEAAEWDRLH PVHAGPIAPG QMREPRGSDI AGTTSTLQEQ

IAWMTSNPPI²⁵⁰

PVGDIYKRWI ILGLNKIVRM YSPVSILDIK QGPKEPFRDY

VDRFFKTLRA³⁰⁰

EQATQDVKNW MTD³¹³

According to another aspect of the invention a consensus amino acid sequence for the partial pol gene of HIV-1 subtype C is the following:

LTEEKIKALT AICEEMEKEG KITKIGPENP YNTPVFAIKK

KDSTKWRKL⁵⁰

VDFRELNKRT QDFWEVQLGI PHPAGLKKKK SVTVLDVGDA

YFSVPLDEGF¹⁰⁰

RKYTAFTIPS INNETPGIRY QYNVLPQGWK GSPAIFQSSM

TKILEPFRAK¹⁵⁰

NPEIVIYQYM DDLYVGSDLE IGQHRAKIEE LREHLLKWGF

TTPDKKHQKE²⁰⁰

PPFLWMGYEL HPDKWTVQPI QLPEKDSWTV NDIQKLVGKL

NWASQIYPGI²⁵⁰

KVRQLCKLLR GAKALTDIVP LTEEAELE²⁷⁸

According to another aspect of the invention a consensus amino acid sequence for the partial env gene of HIV-1 subtype C is the following:

YCAPAGYAIL KCNNKTFNGT GPCNNVSTVQ CTHGIKPVVS

TQLLLNGSLA⁵⁰

EEEIIIRSEN LTNNAKTIIV HLNESVEIVC TRPNNNTRKS

IRIGPGOTFY¹⁰⁰

ATGDIIGDIR QAHCNISEGK WNKTLQKVKK KLKEELYKYK

VVEIKPLGIA¹⁵⁰

PTEAKRRVVE REKRAVGIGA VFLGFLGAAG STMGAASITL

TVQARQLLSG²⁰⁰

IVQQQSNLLR AIEAQQHMLQ LTVWGIKQL²²⁹

### DESCRIPTION OF THE DRAWINGS

FIG. 1 shows a schematic representation of the HIV-1 genome and illustrates the location of overlapping fragments that were sequenced having been generated by reverse transcriptase followed by polymerase chain reaction, in order to generate the South African consensus sequence;

FIG. 2 shows a phylogenetic tree of nucleic acid sequences of various HIV-1 subtype C isolates based on the (partial) sequences of the gag gene of the various isolates and includes a number of consensus sequences as well as the South African consensus sequence of the present invention and a selected isolate, Du422, of the present invention;

FIG. 3 shows a phylogenetic tree of nucleic acid sequences of various HIV-1 subtype C isolates based on the (partial) sequences of the pol gene of the various isolates and includes a number of consensus sequences as well as the South African consensus sequence of the present invention and a selected isolate, Du152, of the present invention;

FIG. 4 shows a phylogenetic tree of nucleic acid sequences of various HIV-1 subtype C isolates based on the (partial) sequences of the env gene of the various isolates and includes a number of consensus sequences as well as the South African consensus sequence of the present invention and a selected isolate, Du151, of the present invention

FIG. 5 shows how the sequences of the gag genes of each of a number of isolates varies from the South African consensus sequence for the gag gene which was developed according to the present invention;

FIG. 6 shows how the sequences of the pol genes of each of a number of isolates varies from the South African consensus sequence for the pol gene which was developed according to the present invention;

FIG. 7 shows how the sequences of the env genes of each of a number of isolates varies from the South African consensus sequence for the env gene which was developed according to the present invention;

FIG. 8 shows a phylogenetic tree of amino acid sequences of various HIV-1 subtype C isolates based on the sequences of the (partial) gag gene of the various isolates and includes a number of consensus sequences as well as the South African consensus sequence of the present invention and a selected isolate, Du422, of the present invention;

FIG. 9 shows a phylogenetic tree of amino acid sequences of various HIV-1 subtype C isolates based on the sequences of the (partial) pol gene of the various isolates and includes a Cpol consensus sequence as well as a South African consensus sequence of the present invention and a selected isolate, Du151, of the present invention;

FIG. 10 shows a phylogenetic tree of amino acid sequences of various HIV-1 subtype C isolates based on the sequences of the (partial) env gene of the various isolates and includes a Cenv consensus sequence as well as a South African consensus sequence of the present invention and a selected isolate, Du151, of the present invention;

FIG. 11 shows the percentage amino acid sequence identity of the sequenced gag genes of the various isolates in relation to one another, to the gag clone and to the South African consensus sequence for the gag gene and is based on a pairwise comparison of the gag genes of the isolates;

FIG. 12 shows the percentage amino acid sequence identity of the sequenced pol genes of the various isolates in relation to one another, to the pol clone and to the South African consensus sequence for the pol gene and is based on a pairwise comparison of the pol genes of the isolates;

FIG. 13 shows the percentage amino acid sequence identity of the sequenced env genes of the various isolates in relation to one another, to the env clone and to the South African consensus sequence for the env gene and is based on a pairnvise comparison of the env genes of the isolates;

FIG. 14 shows a phylogenetic tree analysis of nucleic acid sequences of various HIV-1 subtype C isolates (or vaccine strains) based on the complete sequences of the gag genes of the various isolates and shows the gag gene from a selected isolate, Du422, of the present invention compared to the other subtype C sequences;

FIG. 15 shows a phylogenetic tree analysis of nucleic acid sequences of various HIV-1 subtype C isolates (or vaccine strains) based on the complete sequences of the pol genes of the various isolates and shows the pol gene from a selected isolate, Du151, of the present invention compared to the other subtype C sequences;

FIG. 16 shows a phylogenetic tree analysis of nucleic acid sequences of various HIV-1 subtype C isolates (or vaccine strains) based on the complete sequences of the env gene of the various isolates and shows the env gene from a selected isolate, Du151, of the present invention compared to the other subtype C sequences; and

### LIST OF SEQUENCES

Sequence I.D. No 1 shows the nucleic acid sequence (cDNA) of the sequenced gag gene of the isolate Du422;

Sequence I.D. No 2 shows the amino acid sequence of the sequenced gag gene of the isolate Du422, derived from the nucleic acid sequence;

Sequence I.D. No 3 shows the nucleic acid sequence (cDNA) of the sequenced pol gene of the isolate Du151;

Sequence I.D. No 4 shows the amino acid sequence of the sequenced pol gene of the isolate Du151, derived from the nucleic acid sequence;

Sequence I.D. No 5 shows the nucleic acid sequence (cDNA) of the sequenced env gene of the isolate Du151;

Sequence I.D. No 6 shows the amino acid sequence of the sequenced env gene of the isolate Du151, derived from the nucleic acid sequence;

Sequence I.D. No 7 shows the nucleic acid sequence (DNA) of the resynthesized sequenced gag gene of the isolate Du422 modified to reflect human codon usage for the purposes of increased expression;

Sequence I.D. No 8 shows the amino acid sequence of the resynthesized sequenced gag gene of the isolate Du422 modified to reflect human codon usage for the purposes of increased expression; Sequence I.D. No 9 shows the nucleic acid sequence (DNA) of the resynthesized sequenced pol gene of the isolate Du151 modified to reflect human codon usage for the purposes of increased expression;

Sequence I.D. No 10 shows the amino acid sequence of the resynthesized sequenced pol gene of the isolate Du151 modified to reflect human codon usage for the purposes of increased expression;

Sequence I.D. No 11 shows the nucleic acid sequence (DNA) of the resynthesized sequenced env gene of the isolate Du151 modified to reflect human codon usage for the purposes of increased expression;

Sequence I.D. No 12 shows the amino acid sequence of the resynthesized sequenced env gene of the isolate Du151 modified to reflect human codon usage for the purposes of increased expression;

Sequence I.D. No 13 shows the nucleic acid sequence (cDNA) of the sequenced env gene of the isolate Du179; and

Sequence I.D. No 14 shows the amino acid sequence of the sequenced env gene of the isolate Du179.

### DETAILED DESCRIPTION OF THE INVENTION

This invention relates to the selection of HIV-1 subtype isolates and the use of their genes and modifications and derivatives thereof in making prophylactic and therapeutic pharmaceutical compositions and formulations, and in particular vaccines against HIV-1 subtype C. The compositions could therefore be used either prophylactically to prevent infection or therapeutically to prevent or modify disease. A number of factors must be taken into consideration in the development of an HIV vaccine and one aspect of the present invention relates to a process for the selection of suitable HIV isolates for the development of a vaccine.

The applicant envisages that the vaccine developed according to the above method could be used against one or more HIV subtypes other than HIV-1 subtype C.

An HIV vaccine aims to elicit both a CD8+ cytotoxic T lymphocyte (CTL) immune response as well as a neutralizing antibody response. Many current vaccine approaches have primarily focused on inducing a CTL response. It is thought that the CTL response may be more important as it is associated with the initial control of viral replication after infection, as well as control of replication during disease, and is inversely correlated with disease progression (Koup et al., 1994, Ogg et al., 1999 Schmitz et al., 1999). The importance of CTL in protecting individuals from infection is demonstrated by their presence in highly exposed seronegative individuals such as sex-workers (Rowland-Jones et al., 1998).

Knowledge of genetic diversity is highly relevant to the design of vaccines aiming at eliciting a cytotoxic T-lymphocyte (CTL) response. There are many CTL epitopes in common between viruses, particularly in the gag and pol region of the genome (HIV Molecular Immunology Database, 1998). In addition, several studies have now shown that there is a cross-reactive CTL response: individuals vaccinated with a subtype B-based vaccine could lyse autologous targets infected with a diverse group of isolates (Ferrari et al., 1997); and CTLs from non-B infected individuals could lyse subtype B-primed targets (Betts et al. 1997; Durali et al, 1998). A comparison of CTL epitopes in the HIV-1 sequence database shows about 40% of gp41 and 84% of p24 epitopes are identical or have only one amino acid difference between subtypes. Although this is a very crude analysis and does not take into consideration populations or dominant responses to certain epitopes, it does however indicate that there is a greater conservation of cytotoxic T epitopes within a subtype compared to between subtypes and that there will be a greater chance of a CTL response if the challenge virus is the same subtype as the vaccine strain.

The importance of genetic diversity in inducing a neutralizing antibody response appears to be less crucial. In general, neutralization serotypes are not related to genetic subtype. Some individuals elicit antibodies that can neutralize a broad range of viruses, including viruses of different subtypes while others fail to elicit effective neutralizing antibodies at all (Wyatt and Sodroski, 1998; Kostrikis et al., 1996; Moore et al., 1996). As neutralizing antibodies are largely evoked against functional domains of the virus which are essentially conserved, it is probable that HIV-1 genetic diversity may not be relevant in producing a vaccine designed to elicit neutralizing antibodies. Viral strains used in the design of a vaccine need to be shown by genotypic analysis to be representative of the circulating strains and not an unusual or outlier strain. In addition, it is important that a vaccine strain also has the phenotype of a recently transmitted virus, which is NSI and uses the CCR5 co-receptor.

A process was developed to identify appropriate strains for use in developing a vaccine for HIV-1 subtype C. Viral isolates from acutely infected individuals were collected. They were sequenced in the env, gag and pol regions and the amino acid sequences for the env, gag and pol genes from these isolates were compared. A consensus sequence, the South African consensus sequence, was then formed by selecting the most frequently appearing amino acid at each position. The consensus sequence for each of the gag, pol and env genes of HIV-1 subtype C also forms an aspect of the invention. Appropriate strains for vaccine development were then selected from these isolates by comparing them with the consensus sequence and characterising them phenotypically. The isolates also form an aspect of the invention.

In order to select for NSI strains which use the CCR5 co-receptor, a well established sex worker cohort was used to identify the appropriate strains. Appropriate strains were identified from acutely infected individuals by comparing them with the consensus sequence which had been formed. Viral isolates from fifteen acutely infected individuals were sequenced in the env, gag and pol and phenotypically characterised. These sequences were compared with viral isolates from fifteen asymptomatic individuals from another region having more than 500 CD4 cells and other published subtype C sequences located in the Los Alamos Database (http)://ww.hiv-web.lanl.gov/).

Three potential vaccine strains, designated Du151, Du422 and Du179, were selected. Du 151 and Du 422 were selected based on amino acid homology to the consensus sequence in all three gene regions env, gag and pol, CCR5 tropism and ability to grow and replicate in tissue culture. Du 179 is a R5X4 virus and was selected because the patient in which this strain was found showed a high level of neutralising antibodies. The nucleotide and amino acid sequences of the three gene regions of the three isolates and modifications and derivatives thereof also form aspects of the invention.

The vaccines of the invention will be formulated in a number of different ways using a variety of different vectors. They involve encapsulating RNA or transcribed DNA sequences from the viruses in a variety of different vectors. The vaccines will contain at least part of the gag gene from the Du422 isolate, and at least part of the pol and env genes from the Du151 isolate of the present invention and/or at least part of the env gene from the Du179 isolate of the present invention or derivatives or modifications thereof.

Genes for use in DNA vaccines have been resynthesized to reflect human codon usage. The gag Du422 gene was designed so that the myristylation site and inhibitory sequences were removed. Similarly resynthesized gp 160 (the complete env gene consisting of gp 120 and gp 41) and pol genes will be expressed by DNA vaccines. The gp160 gene sequence has also been changed as described above for the gag gene to reflect human codon usage and the rev responsive element removed. The protease, inactivated reverse transcriptase and start of the RNAse H genes from Du151 pol are optimised for increased expression and will be joined with gag at an inserted Bgl1 site. The gag-pol frameshift will be maintained to keep the natural balance of gag to pol protein expression.

Another vaccine will contain DNA transcribed from the RNA for the gag gene from the Du422 isolate and RNA from the pol and env genes from the Du151 isolate and/or RNA from the env gene from the Du179 isolate. These genes could also be expressed as oligomeric envelope glycoprotein complexes (Progenics, USA) as published in J Virol 2000 Jan;74(2):627-43 (Binley, J. L. et al.), the adeno associated virus (AAV) (Target Genetics) and the Venezuelan equine encephalitus virus (U.S. patent application U.S. S No. 60/216,995, which is incorporated herein by reference).

The Isolation and Selection of Viral Strains for the Design of a Vaccine

The following criteria were used to select appropriate strains for inclusion into HIV-1 vaccines for Southern Africa:

that the strains be genotypically representative of circulating strains;

that the strain not be an outlier strain;

that the strain be as close as possible to the consensus amino acid sequence developed according to the invention for the env, gag and pol genes of HIV-1 subtype C;

that the strain have an R5 phenotype, i.e. a phenotype associated with transmission for selection of the RNA or cDNA to be included for the env region; and

that the vaccine be able to be grown in tissue culture.

The following procedure was followed in the selection of viral strains for the design of a vaccine. A well-established sex worker cohort in Kwazulu Natal, South Africa was used to identify the appropriate strains for use in an HIV vaccine. Viral isolates from 15 acutely infected individuals were sequenced in env, gag and pol and were also isolated and phenotypically characterised. These sequences were compared with a similar collection from asymptomatic individuals from the Gauteng region in South Africa as well as other published subtype C sequences.

Patients

Individuals with HIV infection were recruited from 4 regions in South Africa. Blood samples were obtained from recently infected sex workers from Kwazulu-Natal (n=13). Recent infection was defined as individuals who were previously seronegative and had became seropositive within the previous year. Samples were also collected from individuals attending out-patients clinics in Cape Town (n=2), women attending ante-natal clinics in Johannesburg (n=7) and men attending a STD clinic on a gold mine outside Johannesburg (n=8). The latter 2 groups were clinically stable and were classified as asymptomatic infections. Blood samples were collected in EDTA and used to determine the CD4 T cell count and genetic analysis of the virus. In the case of recent infections a branched chain (bDNA) assay (Chiron) to measure plasma viral load was done, and the virus was isolated. HIV-1 serostatus was determined by ELISA. The results of the CD4 T cell counts and the viral loads on the sex workers were established and information on the clinical status as at date of seroconversion, CD4, and data on the co-receptor usage is set out in Table 1 below.

Virus Isolation

HIV was isolated from peripheral blood mononuclear cells (PBMC) using standard co-culture techniques with mitogen-activated donor PBMC. 2×10⁶ patient PBMC were co-cultured with 2×10⁶ donor PBMC in 12 well plates with 2 ml RPMI 1640 with 20% FCS, antibiotics and 5% IL-2 (Boehringer). Cultures were replenished twice weekly with fresh medium containing IL-2 and once with 5×10⁵/ml donor PBMC. Virus growth was monitored weekly using a commercial p24 antigen assay (Coulter). Antigen positive cultures were expanded and cultured for a further 2 weeks to obtain 40 mis of virus containing supernatant which was stored at −70° C. until use. The results of the isolation of the viruses from the commercial sex workers is also shown in Table 1 below.

Viral Phenotypes

Virus-containing supernatant was used to assess the biological phenotype of viral isolates on MT-2 and co-receptor transfected cell lines. For the MT-2 assay, 500 ul of supernatant was incubated with 5×10⁴ MT-2 cells in PRMI plus 10% FCS and antibiotics. Cultures were monitored daily for syncitia formation over 6 days. U87.CD4 cell expressing either the CCR5 or CXCR4 co-receptor were grown in DMEM with 10% FCS, antibiotics, 500 ug/ml G418 and 1 ug/ml puromycin . GHOST cells expressing minor co-receptors were grown in DMEM with 10% FCS, 500 ug/ml G418, 1 ug/ml puromycin and 100 ug/ml hygromycin. Cell lines were passaged twice weekly by trypsination. Co-receptor assays were done in 12 well plates; 5×10⁴ cells were plated in each well and allowed to adhere overnight. The following day 500 ul of virus containing supematant was added and incubated overnight to allow viral attachment and infection and washed three times the following day. Cultures were monitored on days 4, 8 and 12 for syncitia formation and p24 antigen production. Cultures that showed evidence of syncitia and increasing concentrations of p24 antigen were considered positive for viral growth. The results of co-receptor usage of the viruses from the commercial sex workers is also shown in Table 1.

TABLE 1
COHORT OF ACUTE INFECTIONS FOR SELECTION OF VACCINE CANDIDATES

| | | | Duration of | | | | Co-culture p24 | | |
|---|---|---|---|---|---|---|---|---|---|
| Sample ID | Sero date | Sample date | infection | | CD4 count | Viral load | pos | MT-2 assay | Biotype |
| Du115 | 15 May 1998 | 20 May 1999 | 1 | year | 437* | 7,597* | — | No isolate | — |
| Du123 | 17 Aug. 1998 | 17 Nov. 1998 | 3 | mon | 841 | 19,331 | d6 (50 pg) | NSI | R5 |
| Du151 | 12 Oct. 1998 | 24 Nov. 1998 | 1.5 | mon | 367 | >500.000 | d6 (>1 ng) | NSI | R5 |
| Du156 | 16 Nov. 1998 | 17 Nov. 1998 | <1 | mon | 404 | 22.122 | d6 (>1 ng) | NSI | R5 |
| Du172 | 16 Oct. 1998 | 17 Nov. 1998 | 1 | mon | 793 | 1.916 | d6 (<50 pg) | NSI | R5 |
| Du174 | 6 Oct. 97 | 25 May 1999 | 19.5 | mon | 634* | 9,454* | d14 (>1 ng) | NSI | R5 |
| Du179 | 13 Aug. 97 | 20 May 1999 | 21 | mon | 394* | 1,359* | d7 (<50 pg) | SI | R5x4 |
| Du204 | 20 May 1998 | 20 May 1999 | 1 | year | 633* | 8.734* | d7 (<50 pg) | NSI | R5 |
| Du258 | 3 Jun. 1998 | 22 Jun. 1999 | 1 | year | 433* | 9,114* | — | No isolate | — |
| Du281 | 24 Jul. 1998 | 17 Nov. 1998 | 4 | mon | 594 | 24.689 | d6 (1 ng) | NSI | R5 |
| Du285 | 2 Oct. 1998 | — | — | | 560* | 161* | — | No isolate | — |
| Du368 | 8 Apr. 1998 | 24 Nov. 1998 | 7.5 | mon | 670 | 13,993 | d6 (300 pg) | NSI | R5 |
| Du422 | 2 Oct. 1998 | 28 Jan. 1999 | 4 | mon | 397 | 17,118* | d6 (600 pg) | NSI | R5 |
| Du457 | 17 Aug. 1998 | 17 Nov. 1998 | 3 | mon | 665 | 6,658 | — | No isolate | — |

| | | | | | | | | |
|---|---|---|---|---|---|---|---|---|
| Du467 | 26 Aug. 1998 | — | — | 671 | 19.268 | — | No isolate | — |

*date from Nov 98

Sequencing

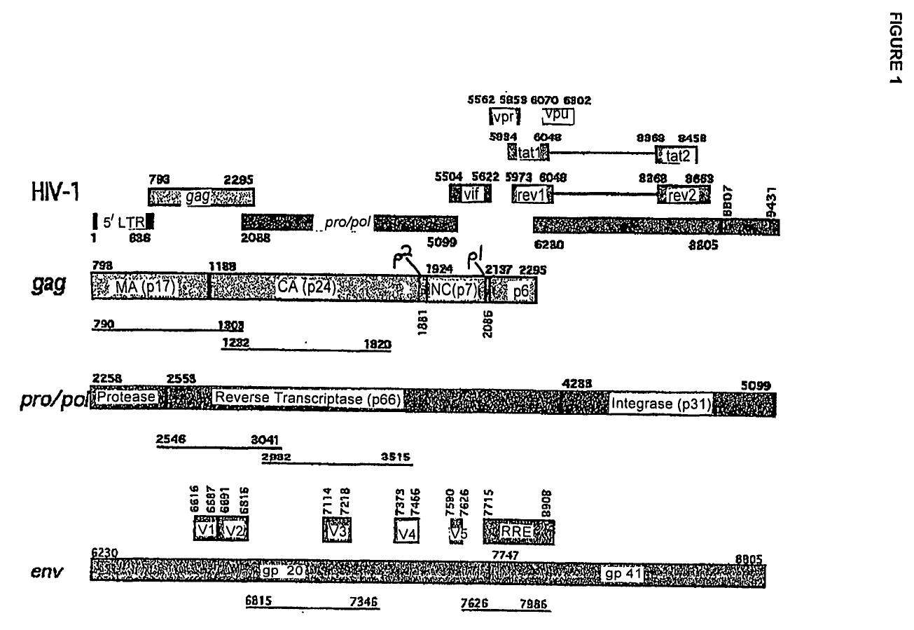

RNA was isolated from plasma and the gene fragments were amplified from RNA using reverse transcriptase to generate a cDNA followed by PCR to generate amplified DNA segments. The positions of the PCR primers are as follows, with the second of each primer pair being used as the reverse transcriptase primer in the cDNA synthesis step (numbering using the HIV-1 HXBr sequence): gag1 (790-813, 1282-1303), gag2 (1232-1253, 1797-1820), pol1 (2546-2573, 3012-3041), pol2(2932-2957, 3492-3515), env1 (6815-6838, 7322-7349), env2 (7626-7653, 7963-7986). The amplified DNA fragments were purified using the QIAQUICK PCR Purification Kit (Qiagen, Germany). The DNA fragments were then sequenced using the upstream PCR primers as sequencing primers. Sequencing was done using the Sanger dideoxyterminator strategy with fluorescent dyes attached to the dideoxynucleotides. The sequence determination was made by electrophoresis using an ABI 377 Sequencer. A mapped illustration of an HIV-1 proviral genome showing the pol, gag and env regions sequenced as described above, is shown in FIG. 1. The following regions were sequenced (numbering according to HXBr, Los Alamos database); 813-1282 (gagl); 1253-1797 (gag2); 2583-3012 (pol1); 2957-3515 (pol2); 6938-7322 (env1); 7653-7963 (env2), as illustrated in FIG. 1.

Genotypic Characterisation

To select the vaccine isolate or isolates, a survey covering portions of the three major HIV genes gag (313 contiguous codons, 939 bases), pol (278 contiguous codons, 834 bases) and env (229 codons in two noncontigous segments, 687 bases) was done (FIG. 1). The map of FIG. 1 shows the 5′long terminal repeat, the structural and functional genes (gag, pol and env) as well as the regulatory and accessory proteins (vif, tat, rev, nef, vpr and vpu). The gag open reading frame illustrates the regions encoding p17 matrix protein and the p24 core protein and the p7 and p6 nuclearcapsid proteins. The pol open reading frame illustrates the protease (PR) p15, reverse transcriptase (RT) p66 and the Rnase H integrase p51. The env open reading frame indicates the region coding for gp120 and the region coding for gp41.

Of a total of 31 isolates, 14 were from the Durban cohort (DU), 15 were from Johannesburg (GG and RB) and 2 from Cape Town (CT). Of these 30 were sequenced in the gag region, 26 in the pol region and 27 in the env region. The isolates that were sequenced are shown in Table 2.

TABLE 2
LIST OF ISOLATES AND THE REGIONS
GENES SEQUENCED

| Isolate | Gag sequence | Pol sequence | Env sequence |
|---|---|---|---|
| CTSC1 | ✓ | ✓ | — |
| CTSC2 | ✓ | ✓ | — |
| DU115 | ✓ | ✓ | ✓ |
| DU123 | ✓ | — | ✓ |
| DU151 | — | ✓ | ✓ |
| DU156 | ✓ | ✓ | ✓ |
| DU172 | ✓ | ✓ | ✓ |
| DU174 | ✓ | ✓ | ✓ |
| DU179 | ✓ | ✓ | ✓ |
| DU204 | ✓ | ✓ | ✓ |
| DU258 | ✓ | ✓ | ✓ |
| DU281 | ✓ | — | ✓ |
| DU368 | ✓ | ✓ | ✓ |
| DU422 | ✓ | ✓ | ✓ |
| DU457 | ✓ | ✓ | ✓ |
| DU467 | ✓ | — | ✓ |
| GG1 | ✓ | — | — |
| GG10 | ✓ | ✓ | ✓ |
| GG3 | ✓ | ✓ | ✓ |
| GG4 | ✓ | ✓ | ✓ |
| GG5 | ✓ | ✓ | ✓ |
| GG6 | ✓ | ✓ | ✓ |
| RB12 | ✓ | — | ✓ |
| RB13 | ✓ | ✓ | ✓ |
| RB14 | ✓ | ✓ | ✓ |
| RB15 | ✓ | ✓ | — |
| RB18 | ✓ | ✓ | ✓ |
| RB21 | ✓ | ✓ | ✓ |
| RB22 | ✓ | ✓ | ✓ |
| RB27 | ✓ | ✓ | ✓ |
| RB28 | ✓ | ✓ | ✓ |

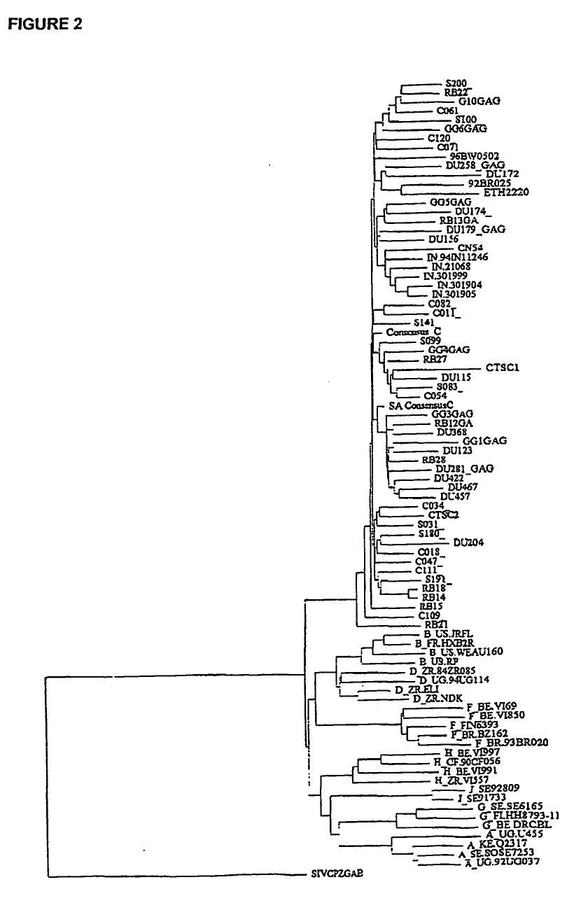

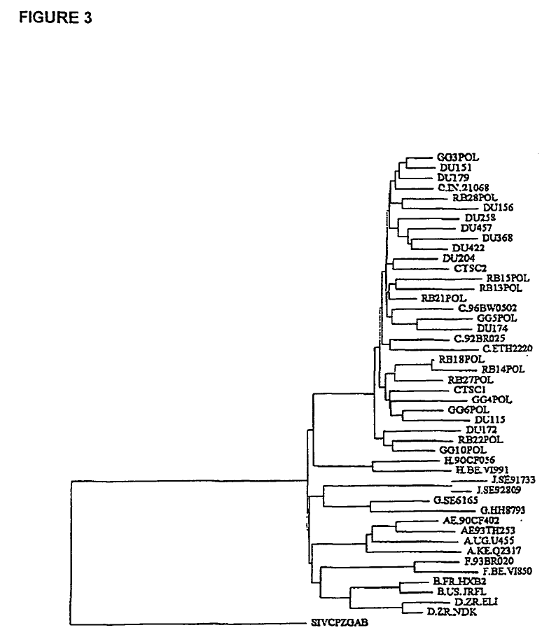

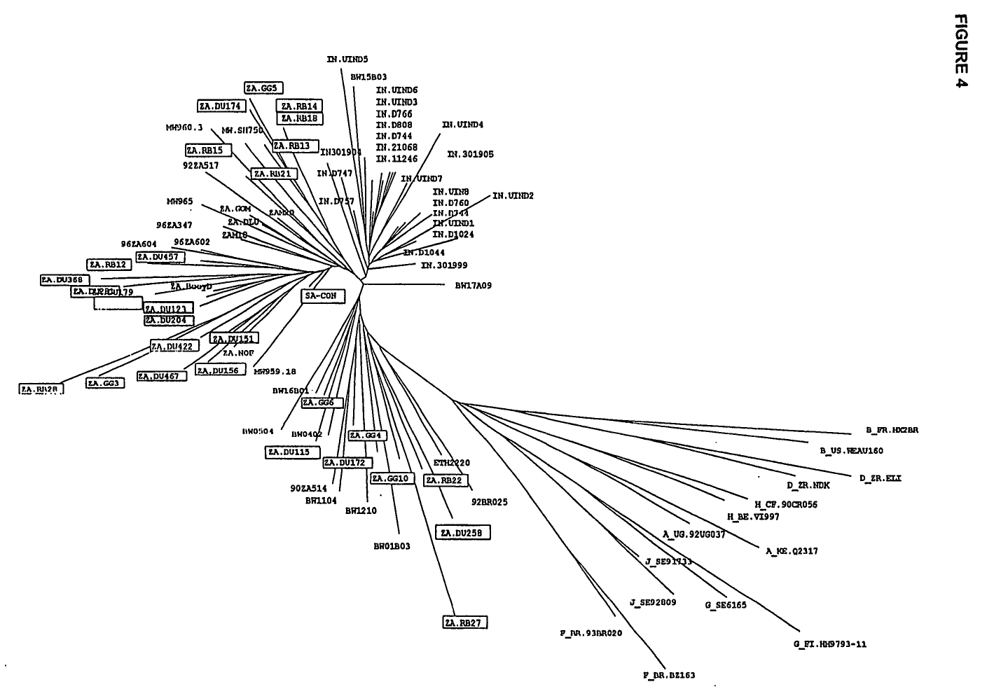

The nucleic acid sequences from the Durban (DU) Johannesburg (GG, RB) and Cape Town (CT) cohorts were phylogenetically compared to all available published subtype C sequences (obtained from the Los Alamos HIV Sequence Database) including sequences from the other southern African countries and the overall subtype C consensus from the Los Alamos HIV sequence database. This comparison was done to ensure that the selected vaccine isolates were not phylogenetic outliers when compared to the Southern African sequences and the results of the comparison are shown in FIG. 2, FIG. 3 and FIG. 4. FIGS. 2 to 4 illustrate that the sequences from Southern Africa are divergent and that the Indian sequences form a separate distinct cluster from these African sequences. The South African sequences are not unique and, in general, are as related to each other as they are to other sequences from Southern Africa. Overall this suggests Indian sequences are unique from Southern African subtype C sequences and that we do not have a clonal epidemic in South Africa, but rather South African viruses reflect the diversity of subtype C viruses in the Southern African region

Determination of a Consensus Sequence

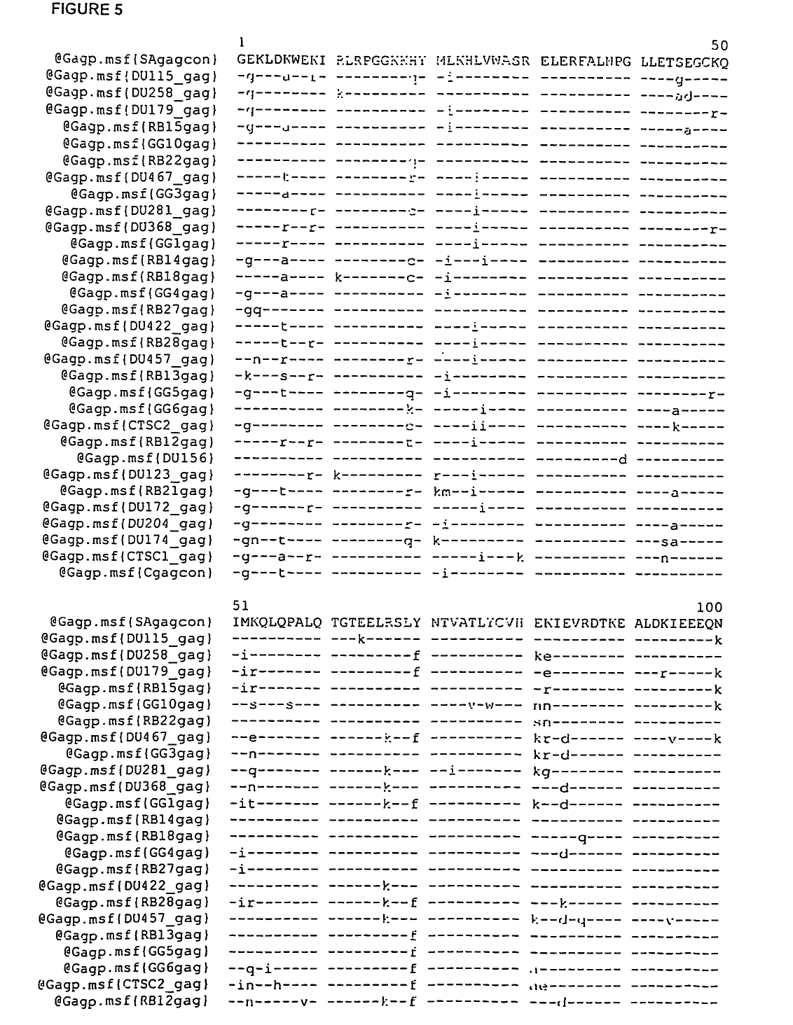

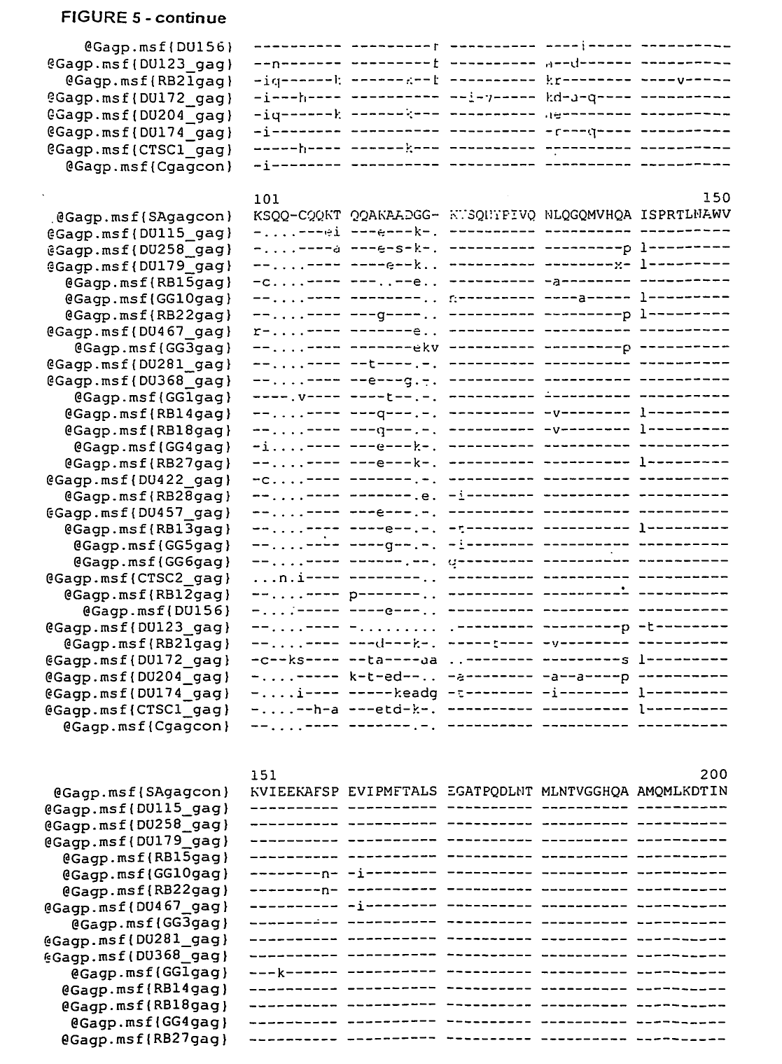

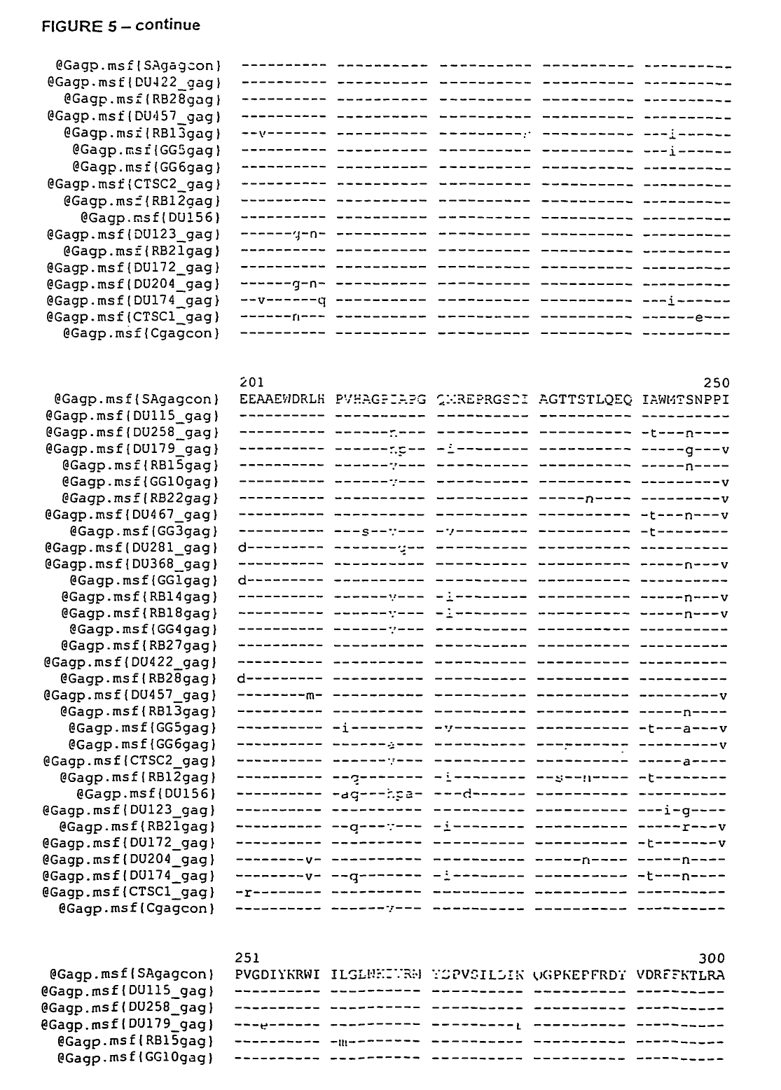

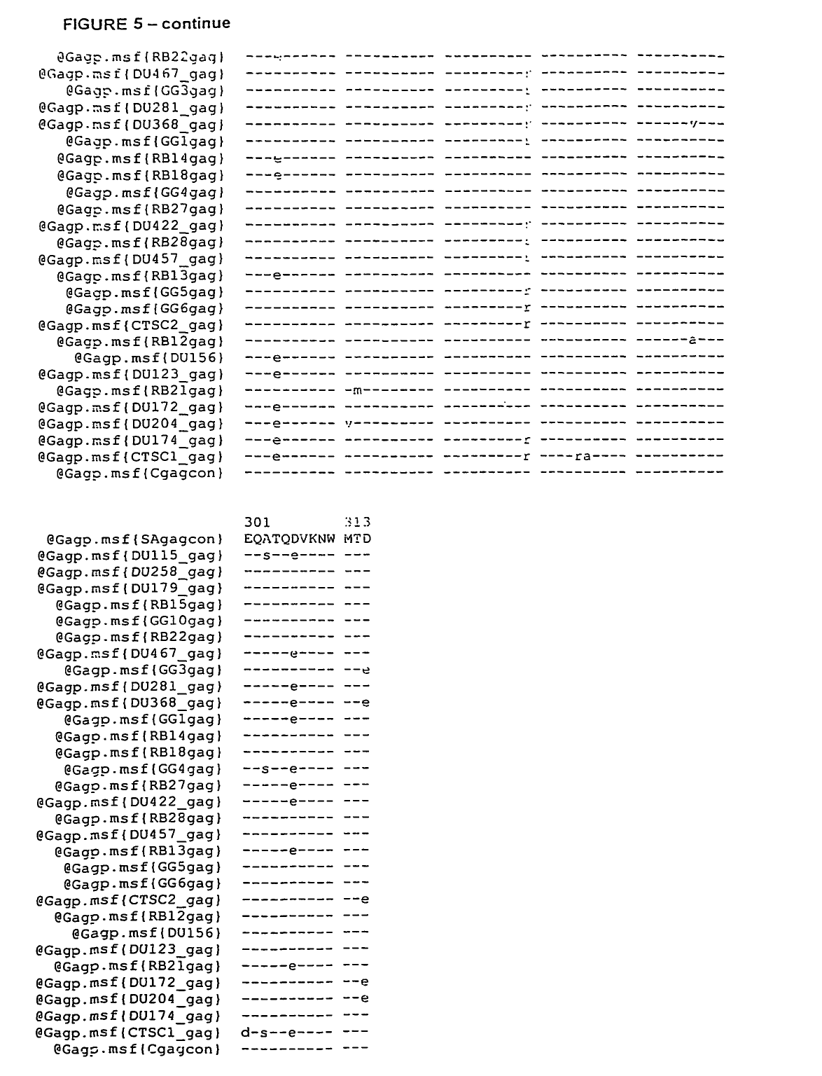

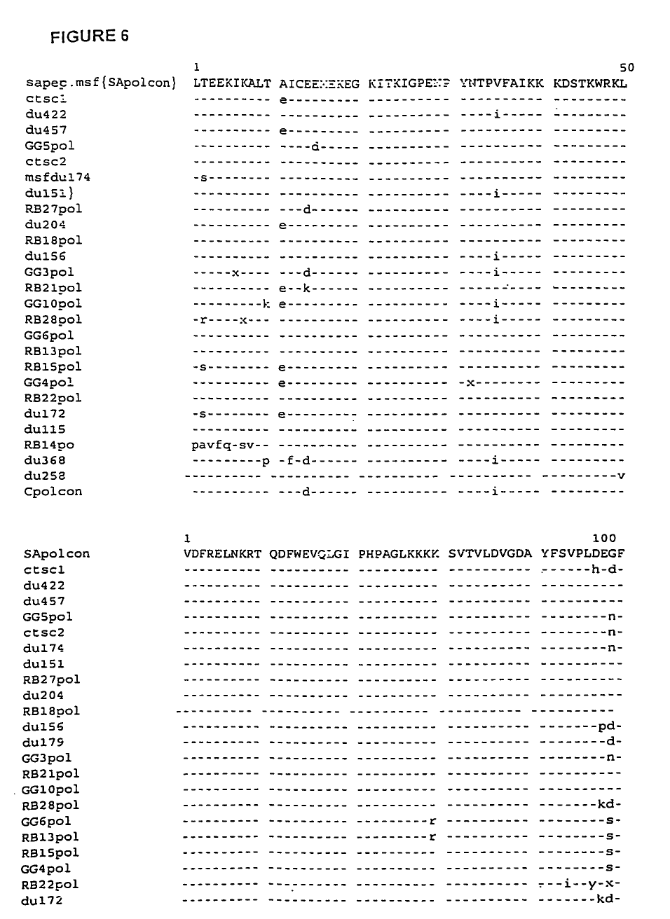

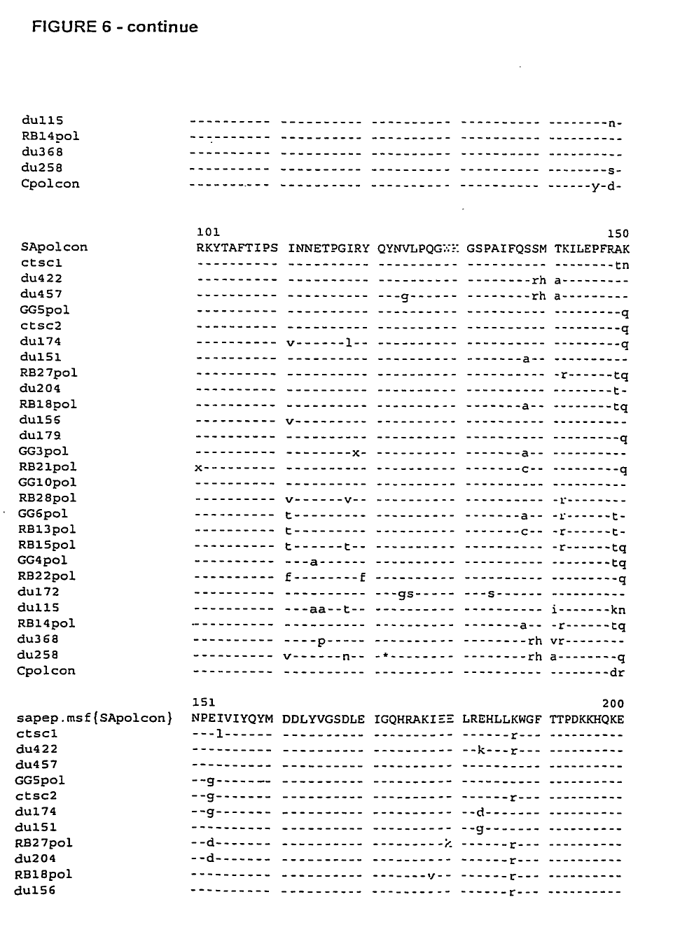

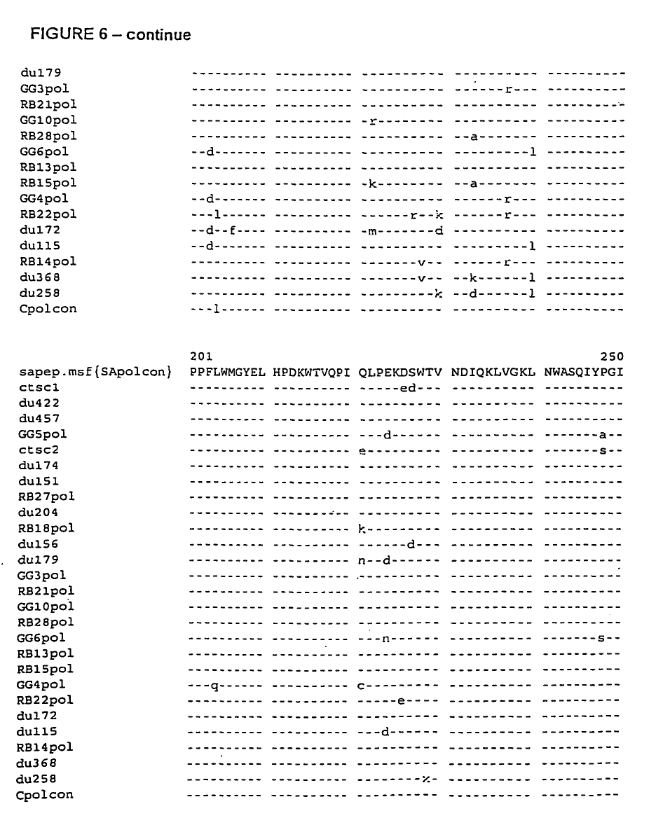

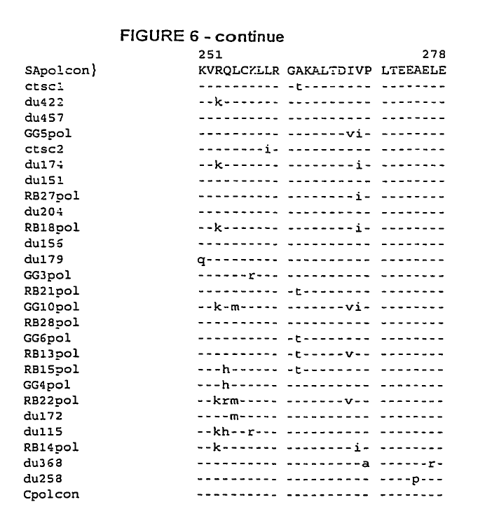

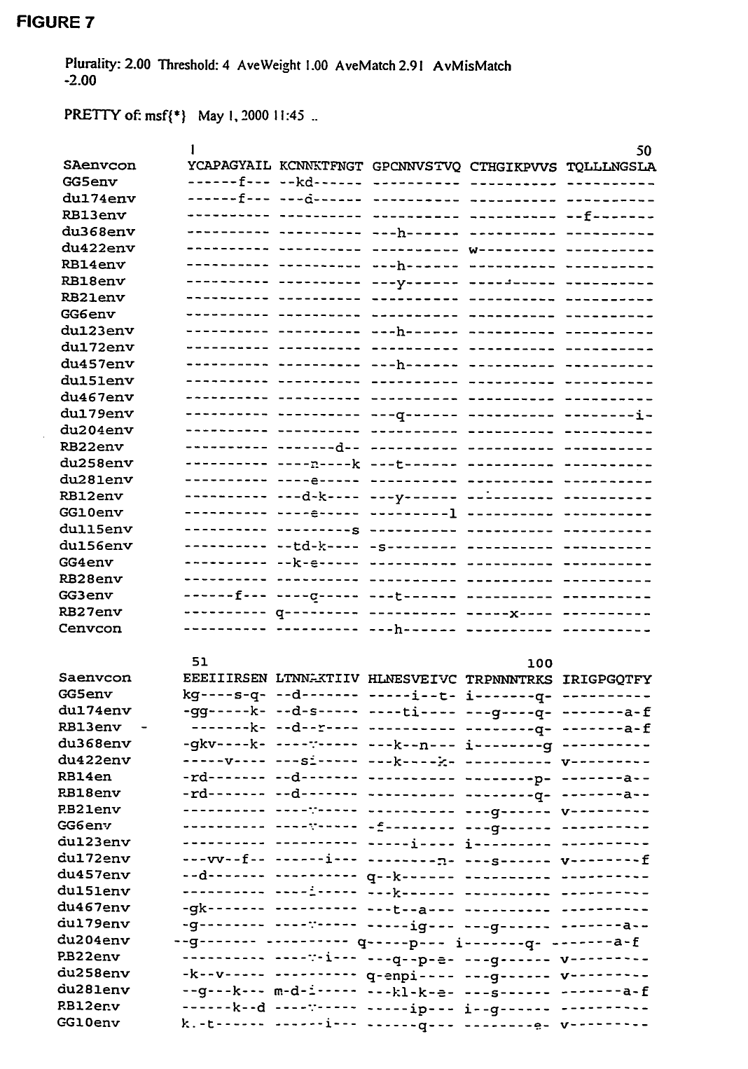

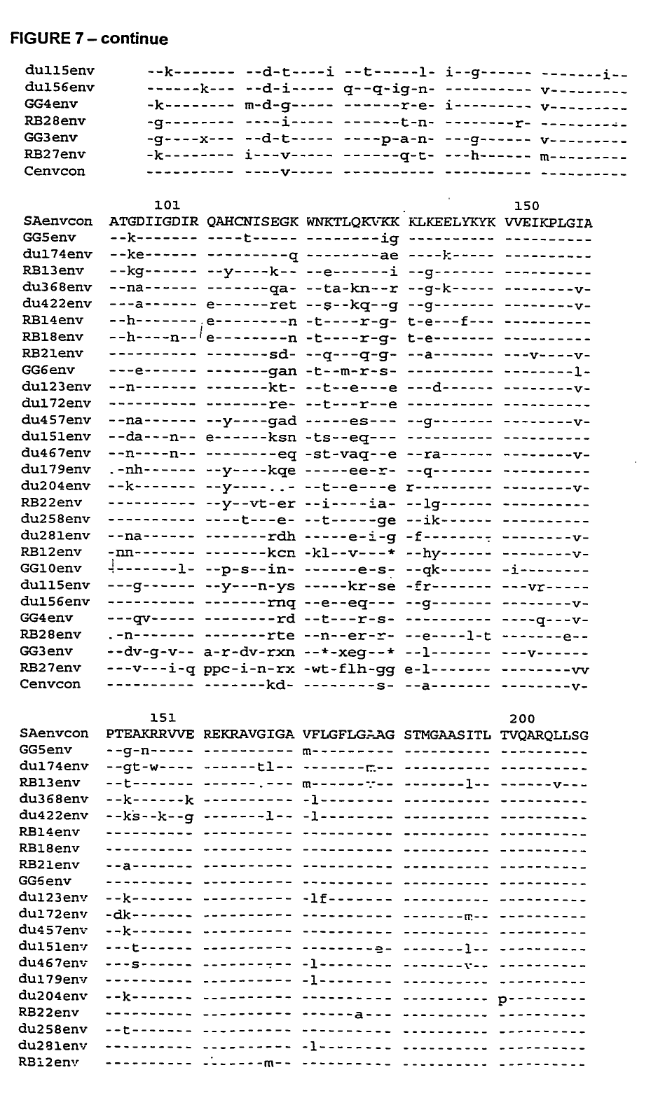

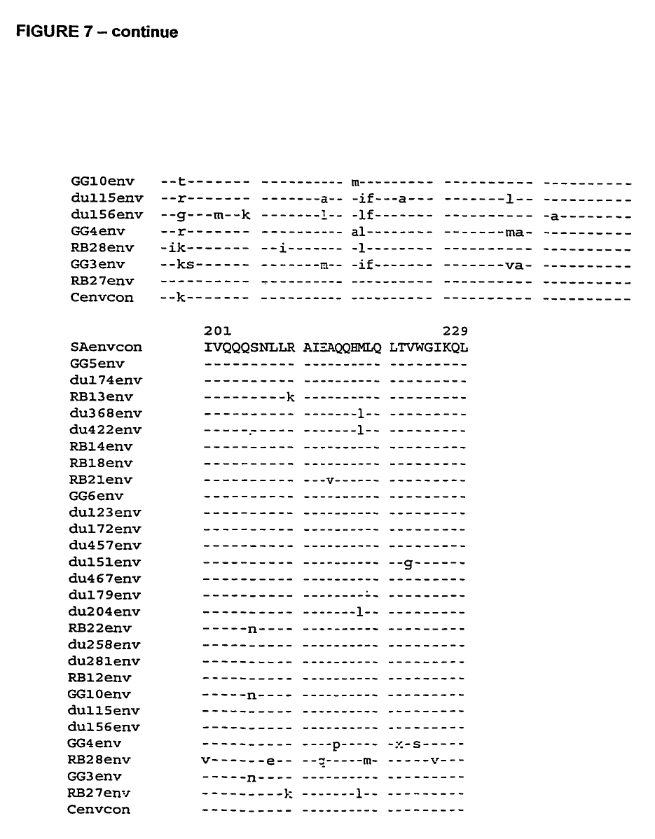

Amino acid sequences were derived from the sequences shown in Table 2 and were used to determine a South African consensus sequence. The most frequently appearing amino acid at each position was selected as the consensus amino acid at that position. In this way, the consensus sequence was determined along the linear length of each of the sequenced gene fragments (gag, pol and env gene fragments). The alignments were done using the Genetics Computer Group (GCG) programs (Pileup and Pretty), which generates a consensus sequence in this manner. These resulted in the consensus sequence for each gene region. The alignments of the amino acid sequences and the resulting consensus sequences are shown in FIGS. 5, 6 and 7.

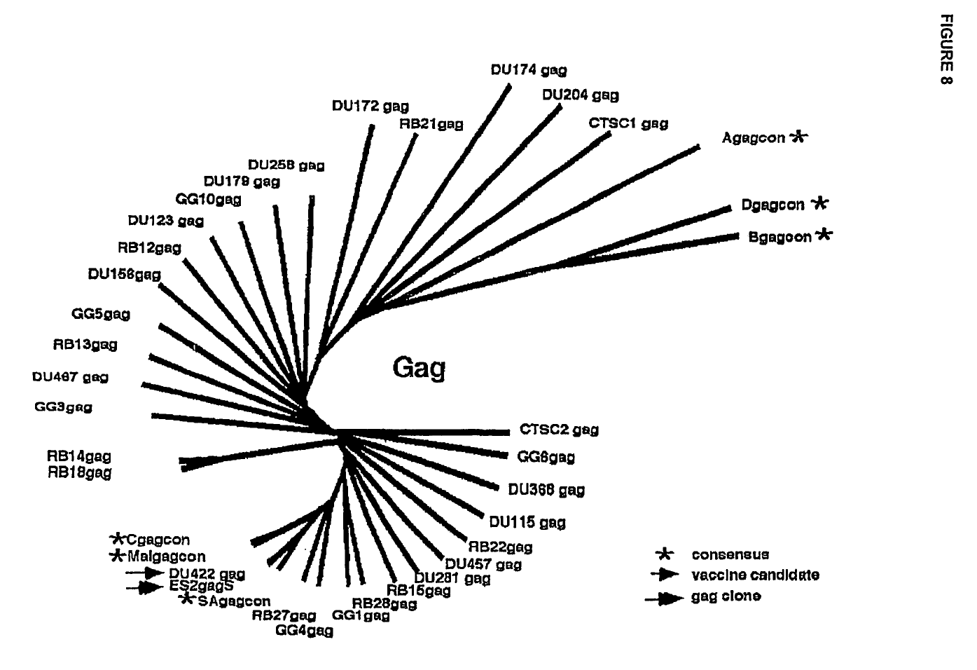

The phylogenetic tree of amino acids showing a comparison of the South African sequences is set out in FIGS. 8, 9 and 10. The ES2 gag S, which is the sequence of the cloned Du422 gag gene, Du151 pol (clone number) 8, which is the sequence of the cloned Du151 pol gene, and Du151 env (clone number) 25, which is the sequence of the cloned Du151 env gene, are vaccine clones. It can be seen from FIGS. 8, 9 and 10 that they are the same as the original isolates. These phylogenetic trees compare the relationship between the HIV proteins. South African isolates were compared with subtype A, B, C and D consensus sequences as well as with the South African consensus (Sagagcon) derived from the South African sequences, a Malawian consensus (Malgagcon) derived from Malawian sequences and overall consensuses (Cgagcon, Cpolcon and Cenvcon) derived from all subtype C sequences on the Los Alamos database.

The final choice of which isolate or isolates to use was based on the similarity of the sequence of the gag, env and pol genes of a particular isolate to the South African consensus sequence which had been derived as set out above as well as the availability of an R5 isolate which had good replication kinetics as shown in Table 1.

Selection of Vaccine Isolates

Based on the considerations and methodology set out above, three strains were selected from the acute infection cohort as the vaccine strains. The first strain is Du422 for the gag gene, the second strain is Du151 for the pol and env genes and the third strain is Du179 which is a possible alternative for the env gene. These three strains were selected for the following reasons.

1. At the time the samples were obtained, Du151 had been infected for 6 weeks and had a CD4 count of 367 cells per ul of blood and a viral load above 500,000 copies per ml of plasma. Given the high viral load, and the recorded time from infection, it is probable that the individual was still in the initial stages of viraemia prior to control of HIV replication by the immune system.

2. At the time the samples were obtained, Du422 had been infected for 4 months with a CD4 count of 397 cells per ul of blood and a viral load of 17,118 copies per ml of plasma. In contrast to Du151 this individual had already brought viral replication under control to a certain extent.

3. At the time the samples were obtained, Du179 had been infected for 21 months with a CD4 count of 394 cells per ul of blood and a viral load of 1,359 copies per ml of plasma.

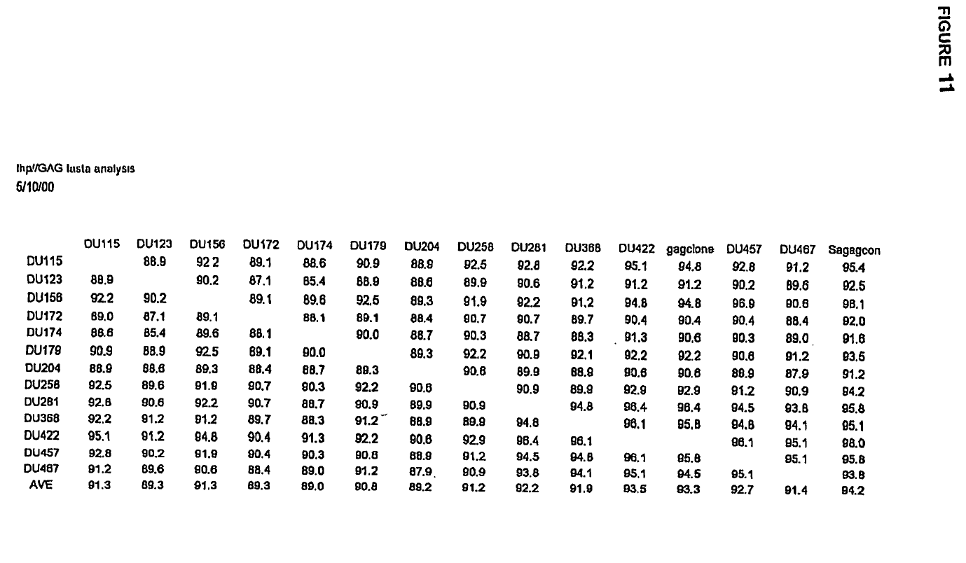

Based on the analysis of the phylogenetic tree shown in FIG. 8 showing the relationship between full length gp120 sequence and other isolates, and the amino acid pairwise comparison shown in FIG. 11, the Du422 gag sequence was shown to be most similar to the South African consensus sequence shown in FIGS. 2 and 5. It shared 98% amino acid sequence identity with the consensus sequence. In addition, the average pairwise distance, which is the percentage difference between the DNA sequences, between the DU422 gag sequence and the other sequences from the seroconverters was the highest of any sequence derived from this cohort, at 93.5%, and nearly as high as the average distance of the isolates to the SA consensus sequence (94.2%). The Du422 gag gene was cloned and the specific clone gave values very similar to the original isolate: having a pairwise identity value with the SA consensus of (98%) and nearly as high an average identity value with the other isolates as the DU422 isolate (93.3%). Thus, both the original DU422 isolate sequence and the generated clone had the highest pairwise percentage similarity to other isolates with the minimal values all being above 90%.

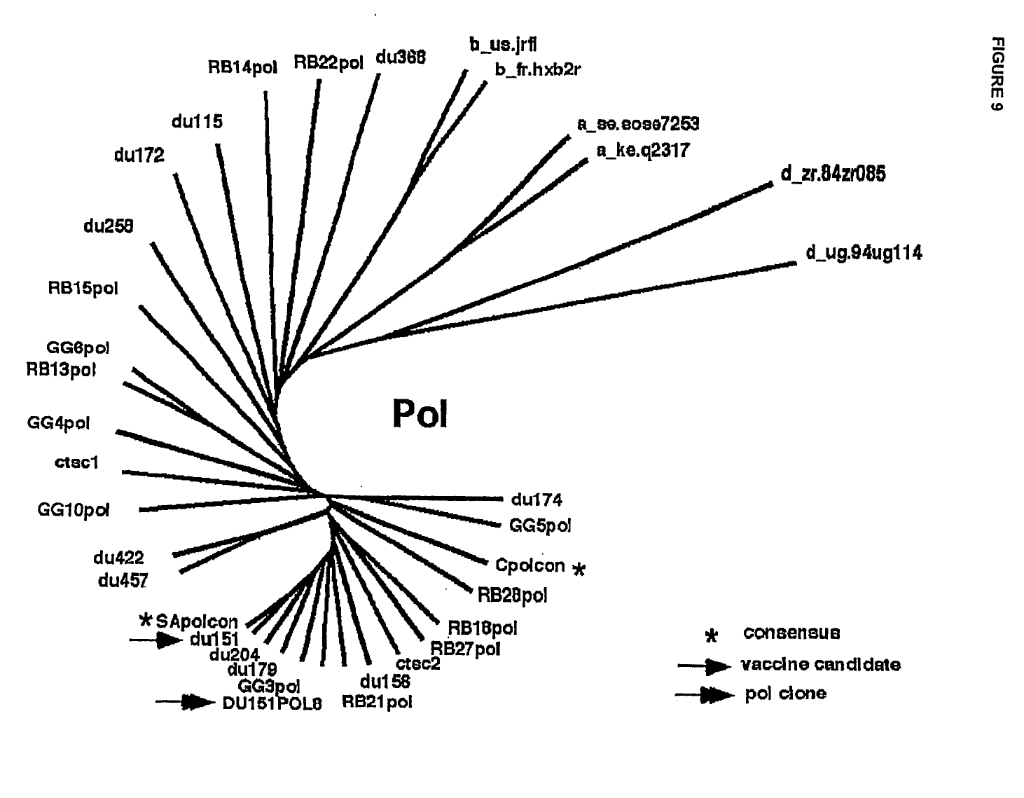

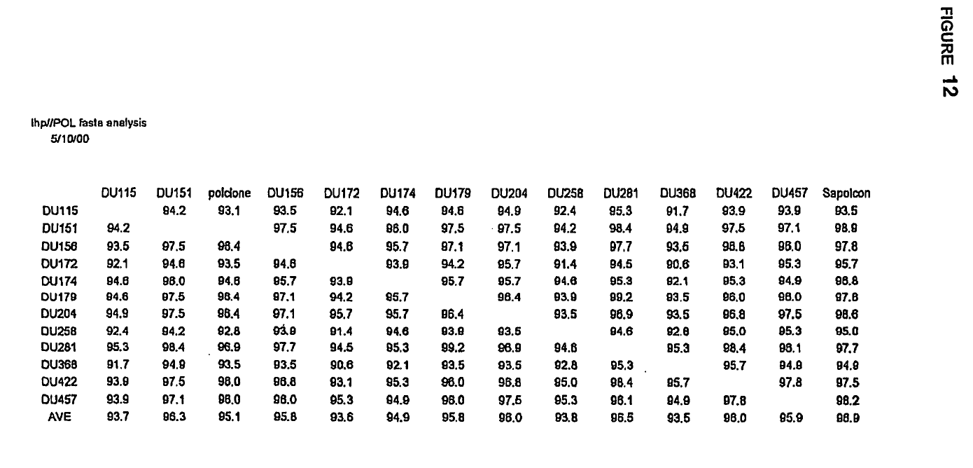

The pol sequences showed the highest values for the pairwise comparisons. Based on the analysis of the phylogenetic tree shown in FIG. 9 and the pairwise identity score with the SA consensus (98.9%) shown in FIG. 12, we chose the DU151 isolate as the source of the pol gene. Other contributing factors in this decision were that this is the same isolate that was chosen for the source of the env gene and that this was an isolate with excellent growth properties in vitro. The actual pol gene clone from the DU151 isolate was somewhat more divergent from the SA consensus sequence (97.8%), and had a smaller average identity score when compared to the other isolates (95.1%). However, we judged the small increase in distance from the consensus not to be significant in this otherwise well conserved HIV-1 gene and therefore chose the DU151 pol gene for further development. Only one of the recent seroconverter sequences was less than 93% identical with the DU151 pol gene segment.

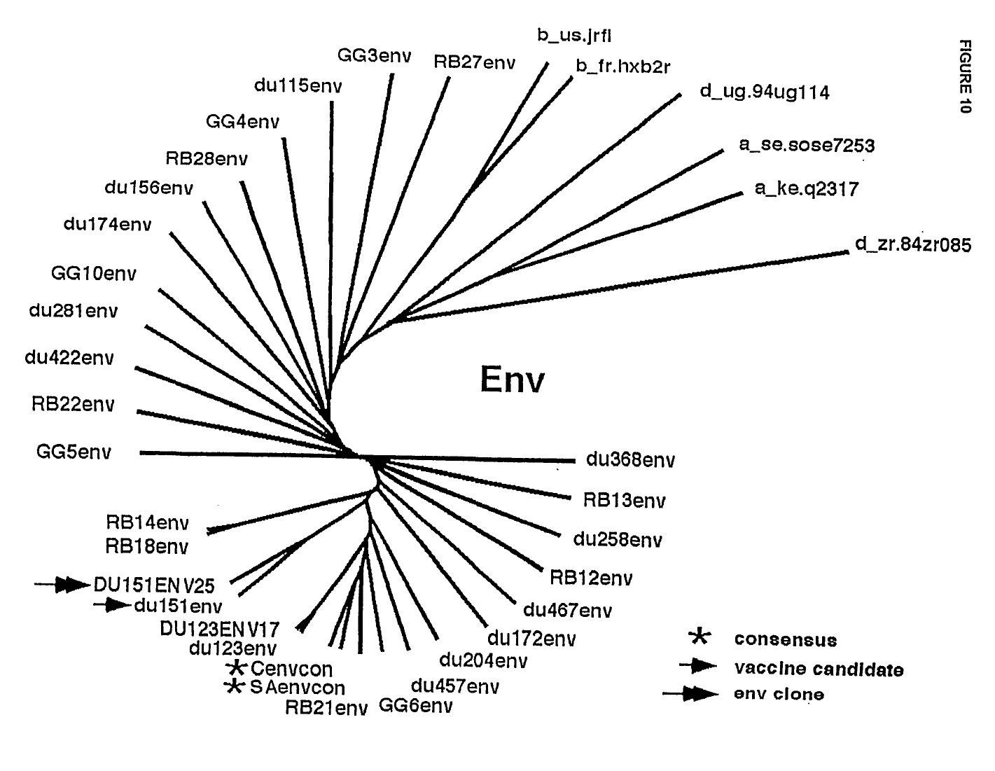

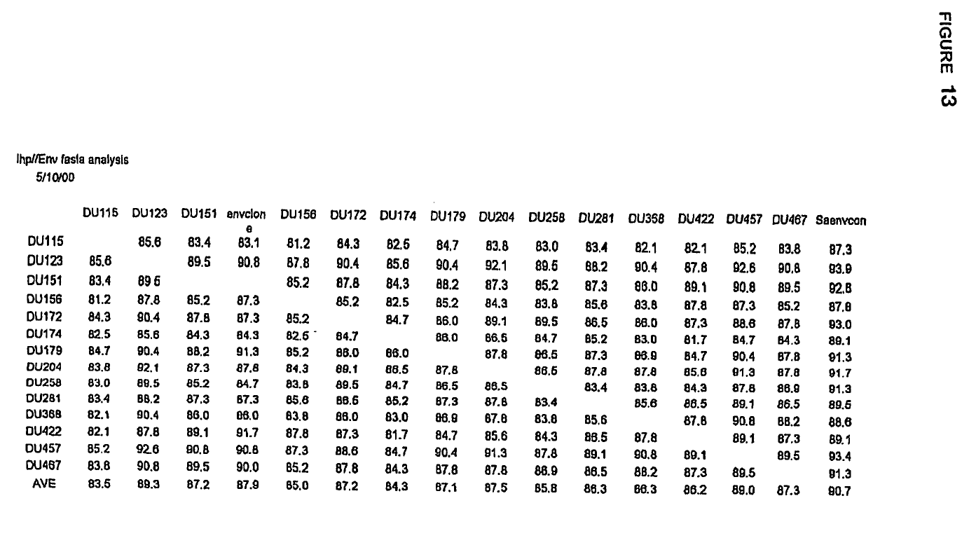

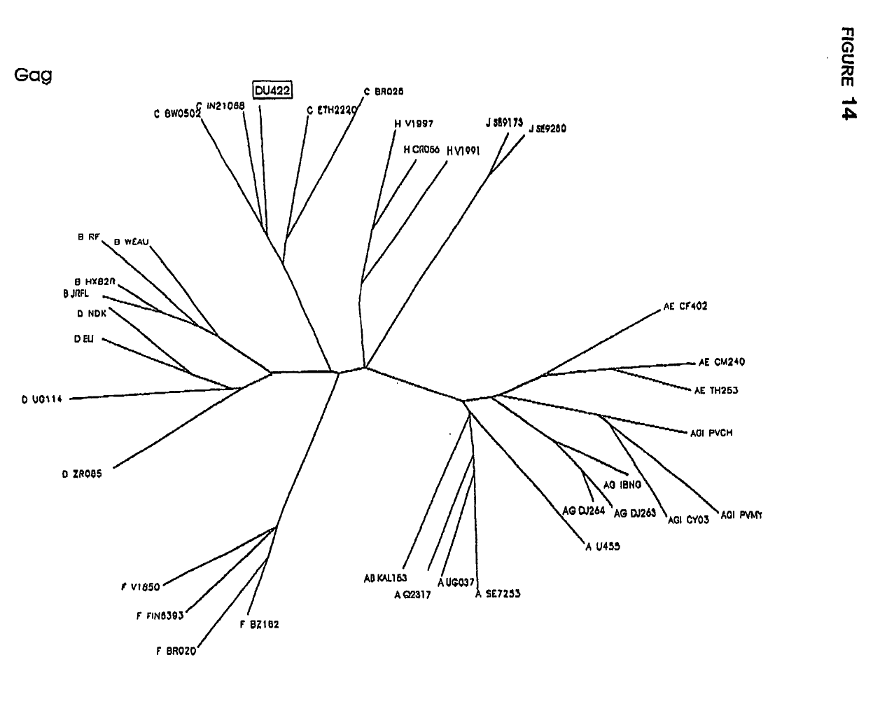

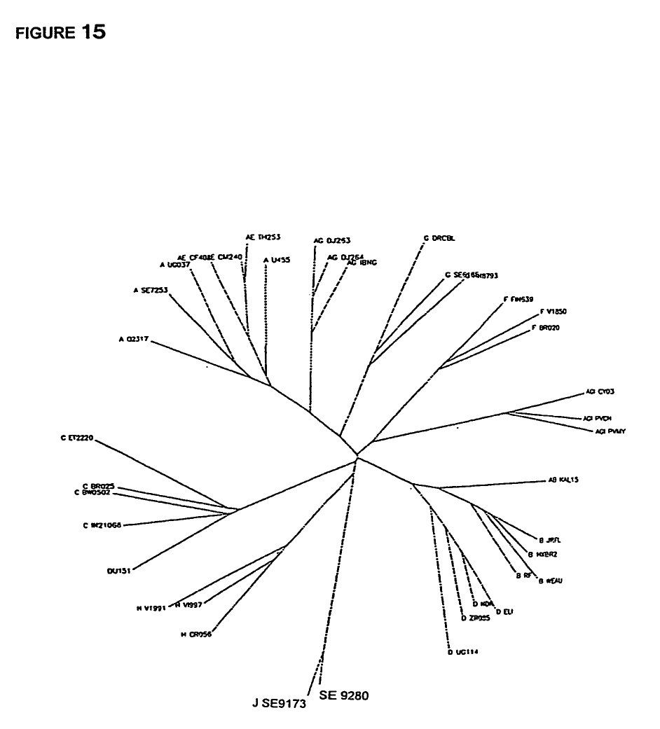

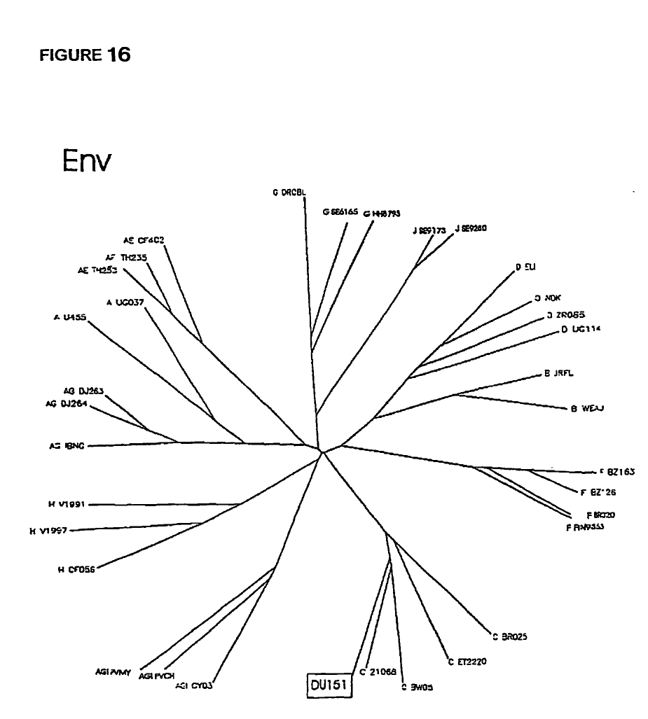

The env gene showed the greatest sequence diversity. Based on the analysis of the phylogenetic tree shown in FIG. 10, we chose the DU151 env gene. The DU151 env gene segment shows an average pairwise comparison score with the other isolates of 87.2%, with the clone being slightly higher (87.9%). The DU151 isolate gene segment has a pairwise identity score of 92.6% with the SA consensus while the DU151 clone is at 91.3%. Finally, all pairwise identity scores are above 83% with either the DU151 isolate sequence or the clone when compared to the other recent seroconverters, as shown in FIG. 13. These pairwise scores make the DU151 sequence similar to the best scores in this sequence pool and combine these levels of similarity with an R5 virus with good cell culture replication kinetics.

The clones representing the full length gene for each of the above viral genes were generated by PCR. Viral DNA present in cells infected with the individual isolates were used for the pol and env clones, and DNA derived directly from plasma by RT-PCR was used for the gag clone. Total DNA was extracted from the infected cell pellets using the QIAGEN DNeasy Tissue Kit. This DNA was used in PCR reactions using the following primers (HXBR numbering, Los Alamos database) in a nested PCR amplification strategy:

gag: outer,623-640, and 2391-2408. inner, 789-810 and 2330-2350

pol: outer,2050-2073, and 5119-5148. inner,2085 -2108, and 5068-5094.

env: outer, 6195-6218, and 8807-8830. inner, 6225-6245, and 8758-8795.

The PCR products were blunt-end cloned into pT7Blue using the Novagen pT7Blue Blunt Kit. The inserts were characterized by doing colony PCR to identify clones with gene inserts. The identity of the insert was confirmed by sequencing the insert on both strands and comparing this sequence to the original sequence.

Modification of Clones

Several modifications were introduced to the cloned genes, as shown in FIGS. 23 to 28. In order to increase levels of expression of proteins, the DNA sequence was resynthesized and the following modifications were made:

the codon usage was changed to reflect human codon usage for increased expression; and

the inhibitory and rev responsive elements were also removed.

The modifications to the gag gene sequence of Du422 are shown in Sequence I.D. numbers 7 and 8.

Also for the DNA, modified vaccinia ankara (MVA) and BCG vaccines, the pol gene was truncated so that only the protease, reverse transcriptase and RNAse H regions of the pol gene will be expressed. In addition, the active site amino acid motive YMDD has been mutated to YMM so that the expressed reverse transcriptase will be catalytically inactive. The modifications to the pol gene of Du151 are shown in sequence I.D. numbers 9 and 10.

Synthetic Genes

The complete gag and env genes were resynthesized to optimise the codons for expression in human cells, also shown in Sequence I.D. numbers 9 to 12. During this process the inhibitory sequences (INS) and rev responsive elements (RRE) are removed which has reported to result in increased expression. The gag gene myristylation signal was mutated as described above and as shown in Sequence I.D. numbers 7 and 8.

The following material has been deposited with the European Collection of Cell Cultures, Centre for Applied Microbiology and Research, Salisbury, Wiltshire SP4 OJG, United Kingdom (ECACC).

Deposits

| Material | ECACC Deposit No. | Deposit Date |
|---|---|---|
| HIV-1 Viral isolate Du151 | Accession Number | 27 Jul. 2000 |
| | 00072724 | |
| HIV-1 Viral isolate Du179 | Accession Number | 27 Jul. 2000 |
| | 00072725 | |
| HIV-1 Viral isolate Du422 | Provisional Accession | 27 Jul. 2000 |
| | Number 00072726 | |
| | Provisional Accession | 22 Mar. 2001 |
| | Number 01032114 | |

The deposit was made under the provisions of the Budapest Treaty on the International Recognition of the Deposit of Microorganisms for the Purpose of Patent Procedure and regulations thereunder (Budapest Treaty).

### References

UNAIDS. AIDS epidemic update. December 1999. www.unaids.org/hivaidsinfo/documents.html

Binley J M, Sanders R W, Clas B, Schuelke N, Master A, Guo Y, Kajumo F, Anselma D J, Maddon P J, Olson W C, Moore J P., J Virol 2000 Jan;74(2):627-43

Bjorndal, A., Sonnerborg, A., Tscherning, C., Albert, J. & Fenyo, E. M. (1999). Phenotypic characteristics of human immunodeficiency virus type 1 subtype C isolates of Ethiopian.

Connor, R., Sheridan, K., Ceraldini, D., Choe, S. & Landau, N. (1997). Changes in co-receptor use correlates with disease progression in HIV-1-infected individuals. J Exp Med 185, 621-628.

Durali D, Morvan J, Letourneur F, Schmitt D, Guegan N, Dalod M, Saragosti S, Sicard D, Levy J P & Gomard E (1998). Cross-reactions between the cytotoxic T-lymphocyte responses of human immunodeficiency virus-infected African and European patients. J Virol 72:3547-53.

Ferrari G, Humphrey W, McElrath M J, Excler J L, Duliege A M, Clements M L, Corey L C, Bolognesi D P & Weinhold K J (1997). Clade B-based HIV-1 vaccines elicit cross-clade cytotoxic T lymphocyte reactivities in uninfected volunteers. Proc Natl Acad Sci U S A 18;94(4): 139-6401.

HIV Molecular Immunology Database 1998: Korber B, Brander C, Koup R, Walker B, Haynes B, & Moore J, Eds. Theoretical Biology and Biophysics Group, Los Alamos National Laboratory, Los Alamos, N.M.

Kostrikis, L. G., Cao, Y., Ngai, H., Moore, J. P. & Ho, D. D (1996). Quantitative analysis of serum neutralization of human immunodeficiency virus type 1 from subtypes A, B. C, D, E, F, and I: lack of direct correlation between neutralization serotypes and genetic subtypes and evidence for prevalent serum-dependent infectivity enhancement. J. Virol. 70, 445-458.

Koup R A, Safrit J T, Cao Y, Andrews C A, McLeod G, Borkowsky W, Farthing C, Ho D D (1994). Temporal association of cellular immune responses with the initial control of viremia in primary human immunodeficiency virus type 1 syndrome. J Virol. 68(7):4650-5.

Moore J P, Cao Y, Leu J, Qin L, Korber B & Ho D D (1996). Inter- and intraclade neutralization of human imunodeficiency virus type 1: genetic clades do not correspond to neutralization serotypes but partially correspond to gp120 antigenic serotypes. J. Virol. 70, 427-444.

Ogg G S, Kostense S, Klein M R, Jurriaans S, Hamann D, McMichael A J & Miedema F (1999). Longitudinal phenotypic analysis of human immunodeficiency virus type 1-specific cytotoxic T lymphocytes: correlation with disease progression. J Virol; 73(11):9153-60.

Peeters, M., Vincent, R., Perret, J.-L., Lasky, M., Patrel, D., Liegeois, F., Courgnaud, V., Seng, R., Matton, T., Molinier, S. & Delaporte, E. (1999). Evidence for differences in MT2 cell tropism according to genetic subtypes of HIV-1: syncitium-inducing variants seem rare among subtype C HIV-1 viruses. J Acquir Imm Def Synd 20, 115-121.

Richman, D. & Bozzette, S. (1994). The impact of the syncytium-inducing phenotype of human immunodeficiency virus on disease progression. J Inf Dis 169, 968-974.

Robertson D L, Anderson J P, Bradac J A, Carr J K, Foley B, Funkhouser R K, Gao R, Hahn B H, Kalish M L, Kuiken C, Learn G H Leitner T, McCutchan F, Osmanov S, Peeters M, Pieniazek D, Salminen M, Sharp P M, Wolinsky S, Korber B (2000). HIV nomenclature proposal. Science 7;288 (5463):55-6.

Rowland-Jones S L, Dong T, Fowke K R, Kimani J, Krausa P, Newell H, Blanchard T, Ariyoshi K, Oyugi J, Ngugi E, Bwayo J, MacDonald K S, McMichael A J & Plummer F A (1998). Cytotoxic T-cell responses to multiple conserved epitopes in HIV-resistant prostitutes in Nairobi. J. Clin. Invest. 102 (9): 1758-1765.

Scarlatti, G., Tresoldi, E., Bjorndal, A., Fredriksson, R., Colognesi, C., Deng, H., Malnati, M., Plebani, A., Siccardi, A., Littman, D., Fenyo, E. & Lusso, P. (1997). In vivo evolution of HIV-1 co-receptor usage and sensitivity to chemokine-mediated suppression. Nat Med 3, 1259-1265.

Schmitz J E, Kuroda M J, Santra S, Sasseville V G, Simon M A, Lifton M A, Racz P, Tenner-Racz K, Dalesandro M, Scallon B J, Ghrayeb J, Forman M A, Montefiori D C, Rieber E P, Letvin N L, Reimann K A (1999). Control of viremia in simian immunodeficiency virus infection by CD8+ lymphocytes. Science 5;283(5403):857-60.

Summary Report: National HIV sero-prevalence survey of women attending public antenatal clinics in South Africa, 1999 (2000). Department of Health, Directorate: Health Systems Research & Epidemiology, April 2000.

Tscheming, C., Alaeus, A., Fredriksson, R., Bjorndal, A., Deng, H., Littman, D., Fenyo, E. M. & Alberts, J. (1998). Differences in chemokine co-receptor usage between genetic subtypes of HIV-1. Virology 241, 181-188.

Wyatt R and Sodroski J (1998). The HIV-1 envelope glycoproteins: Fusogens, antigens and immunogens. Science, 280 (5371):1884-8.

Wyatt R, Kwong, Desjardins E, Sweet R W, Robinson J, Hendrickson W A & Sodroski J G (1998). The antigenic structure of the HIV gp120 envelope glycoprotein. Nature, 393(6686):705-11.

1. A process for the selection of HIV subtype isolates for use in the development of a prophylactic and/or therapeutic pharmaceutical composition comprising the following steps:
isolating viruses from recently infected subjects;
generating a consensus sequence for at least part of at least one HIV gene by identifying the most common codon or amino acid among the isolated viruses at each position along at least part of the gene;
selecting the isolated virus or viruses with a high sequence identity to the consensus sequence, a phenotype which is associated with transmission for the particular HIV subtype.

2. A process according to claim 1, wherein the isolated virus is of the same subtype as a likely challenge strain.

3. A process according to either of claims 1 or 2, wherein the HIV subtype is HIV-1 subtype C.

4. A process according to claim 3, wherein the phenotype which is associated with transmission is a virus that utilises the CCR5 co-receptor and is non syncitium inducing (NSI).

5. An HIV-1 subtype C isolate, designated Du422 and assigned Provisional Accession Number 01032114 by the European Collection of Cell Cultures.

6. An HIV-1 subtype C isolate, designated Du151and assigned Accession Number. 00072724 by the European Collection of Cell Cultures.

7. An HIV-1 subtype C isolate, designated Du179 and assigned Accession Number. 00072725 by the European Collection of Cell Cultures.

8. A molecule having:
(i) the nucleotide sequence set out in Sequence I.D. No 1;
(ii) an RNA sequence corresponding to the nucleotide sequence set out in Sequence I.D. No 1;
(iii) a sequence which will hybridise to the nucleotide sequence set out in Sequence I.D. No 1 or an RNA sequence corresponding to it, under strict hybridisation conditions;
(iv) a sequence which is homologous to the nucleotide sequence set out in Sequence I.D. No 1 or an RNA sequence corresponding to it; or
(v) a sequence which is a modification or derivative of the sequence of any one of (i) to (iv).

9. A molecule according to claim 8, which has the modified sequence set out in Sequence I.D. No 7.

10. A molecule having:
(i) the nucleotide sequence set out in Sequence I.D. No 3;
(ii) an RNA sequence corresponding to the nucleotide sequence set out in Sequence I.D. No 3;
(iii) a sequence which will hybridise to the nucleotide sequence set out in Sequence I.D. No 3 or an RNA sequence corresponding to it, under strict hybridisation conditions;
(iv) a sequence which is homologous to the nucleotide sequence set out in Sequence I.D. No. 3 or an RNA sequence corresponding to it; or
(v) a sequence which is a modification or derivative of the sequence of any one of (i) to (iv).

11. A molecule according to claim 10, which has the modified sequence set out in Sequence I.D. No. 9.

12. A molecule having:
(i) the nucleotide sequence set out in Sequence I.D. No. 5;
(ii) an RNA sequence corresponding to the nucleotide sequence set out in Sequence I.D. No. 5;
(iii) a sequence which will hybridise to the nucleotide sequence set out in Sequence I.D. No. 5 or an RNA sequence corresponding to it, under strict hybridisation conditions;
(iv) a sequence which is homologous to the nucleotide sequence set out in Sequence I.D. No. 5 or an RNA sequence corresponding to it; or
(v) a sequence which is a modification or derivative of the sequence of any one of (i) to (iv).

13. A molecule according to claim 12, which has the modified sequence set out in Sequence I.D. No. 11.

14. A molecule having:
(i) the nucleotide sequence set out in Sequence I.D. No. 13;
(ii) an RNA sequence corresponding to the nucleotide sequence set out in Sequence I.D. No. 13;
(iii) a sequence which will hybridise to the nucleotide sequence set out in Sequence I.D. No. 13 or an RNA sequence corresponding to it, under strict hybridisation conditions;
(iv) a sequence which is homologous to the nucleotide sequence set out in Sequence I.D. No. 13 or an RNA sequence corresponding to it; or
(v) a sequence which is a modification or derivative of the sequence of any one of (i) to (iv).

15. A molecule according to claim 14, which has a modified sequence which has similar or the same modifications as those set out in Sequence I.D. No. 11 for the env gene of the isolate Du151.

16. A polypeptide having:
(i) the amino acid sequence set out in Sequence I.D. No. 2; or
(ii) a sequence which is a modification or derivative of the amino acid sequence set out in Sequence I.D. No. 2.

17. A polypeptide according to claim 16, wherein the modified sequence is set out in Sequence I.D. No. 8.

18. A polypeptide having:
(i) the amino acid sequence set out in Sequence I.D. No. 4; or
(ii) a sequence which is a modification or derivative of the amino acid sequence set out in Sequence I.D. No. 4.

19. A polypeptide according to claim 18, wherein the modified sequence is that set out in Sequence I.D. No. 10.

20. A polypeptide having:
(i) the amino acid sequence set out in Sequence I.D. No. 6; or
(ii) a sequence which is a modification or derivative of the amino acid sequence set out in Sequence I.D. No. 6.

21. A polypeptide according to claim 20, wherein the modified sequence is that set out in Sequence I.D. No. 12.

22. A polypeptide having:
(i) the amino acid sequence set out in Sequence I.D. No. 14;
(ii) a sequence which is a modification or derivative of the amino acid sequence set out in Sequence I.D. No. 14.

23. A polypeptide according to claim 22, wherein the modified sequence has similar or the same modifications as those set out in Sequence I.D. No. 12 for the amino acid sequence of the env gene of the isolate Du151.

24. A consensus amino acid sequence for the partial gag gene of HIV-1 subtype C which is:
GEKLDKWEKI RLRPGGKKHY MLKHLVWASR ELERFALNPG
LLETSEGCKQ⁵⁰
IMKQLQPALQ TGTEELRSLY NTVATLYCVH EKIEVRDTKE
ALDKIEEEQN¹⁰⁰
KSQQ-CQQKT QQAKAADGG- KVSQNYPIVQ NLQGQMVHQA
ISPRTLNAWV¹⁵⁰
EEKAFSP EVIPMFTALS EGATPQDLNT MLNTVGGHQA
AMQMLKDTIN²⁰⁰
EEAAEWDRLH PVHAGPIAPG QMREPRGSDI AGTTSTLQEQ
IAWMTSNPPI²⁵⁰
PVGDIYKRWI ILGLNKIVRM YSPVSILDIK QGPKEPFRDY
VDRFFKTLRA³⁰⁰
EQATQDVKNW MTD³¹³

25. A consensus amino acid sequence for the partial pol gene of HIV-1 subtype C which is:
LTEEKIKALT AICEEMEKEG KITKIGPENP YNTPVFAIKK
KDSTKWRKL-⁵⁰
VDFRELNKRT QDFWEVQLGI PHPAGLKKKK SVTVLDVGDA
YFSVPLDEGF¹⁰⁰
RKYTAFTIPS INNETPGIRY QYNVLPQGWK GSPAIFQSSM
TKILEPFRAK¹⁵⁰
NPEIVIYQYM DDLYVGSDLE IGQHRAKIEE LREHLLKWGF
TTPDKKHQKE²⁰⁰
PPFLWMGYEL HPDKWTVQPI QLPEKDSWTV NDIQKLVGKL
NWASQIYPGI²⁵⁰
KVRQLCKLLR GAKALTDIVP LTEEAELE²⁷⁸

26. A consensus amino acid sequence for the partial env gene of HIV-1 subtype C which is:
YCAPAGYAIL KCNNKTFNGT GPCNNVSTVQ CTHGIKPVVS
TQLLLNGSLA⁵⁰
EEEIIIRSEN LTNNAKTIIV HLNESVEIVC TRPNNNTRKS
IRIGPGOTFY¹⁰⁰
ATGDIIGDIR QAHCNISEGK WNKTLQKVKK KLKEELYKYK
VVEIKPLGIA¹⁵⁰
PTEAKRRVVE REKRAVGIGA VFLGFLGAAG STMGAASITL
TVQARQLLSG²⁰⁰
IVQQQSNLLR AIEAQQHMLQ LTVWGIKQL²²⁹

27. A process according to claim 1, substantantially as herein described.

28. An HIV-1 subtype C isolate according to claim 5, substantially as herein described.

29. An HIV-1 subtype C isolate according to claim 6, substantially as herein described.

30. An HIV-1 subtype C isolate according to claim 7, substantially as herein described.

31. A molecule according to claim 8, substantially as herein described.

32. A molecule according to claim 10, substantially as herein described.

33. A molecule according to claim 12, substantially as herein described.

34. A molecule according to claim 14, substantially as herein described.

35. A polypeptide according to claim 16, substantially as herein described.

36. A polypeptide according to claim 18, substantially as herein described.

37. A polypeptide according to claim 20, substantially as herein described.

38. A polypeptide according to claim 22, substantially as herein described.

39. A consensus amino acid sequence according to claim 24, substantially as herein described.

40. A consensus amino acid sequence according to claim 25, substantially as herein described.

41. A consensus amino acid sequence according to claim 26, substantially as herein described.
